(19) United States
(12) Patent Application Publication
(10) Pub. No.: US 2026/0241037 A1
(43) Pub. Date: Aug. 20, 2026

(54) COMPOSITIONS AND METHODS RELATING TO AGE-RELATED, NEUROLOGICAL, AND VASCULAR DISEASE CONDITIONS

(71) Applicant: THE BOARD OF TRUSTEES OF THE LELAND STANFORD JUNIOR UNIVERSITY, Stanford, CA (US)

(72) Inventors: Sophia Shi, Stanford, CA (US); Anton Wyss-Coray, Stanford, CA (US); Carolyn Bertozzi, Stanford, CA (US)

(21) Appl. No.: 19/164,439

(22) PCT Filed: Mar. 14, 2024
(86) PCT No.: PCT/US2024/019935

§ 371 Date: Sep. 11, 2025

Related U.S. Application Data
(60) Provisional application No. 63/490,144, filed on Mar. 14, 2023.
(60) Provisional application No. 63/490,383, filed on Mar. 15, 2023.

(51) Int. Cl. A61K 48/00; C12N 15/86

(52) U.S. Cl. CPC A61K 48/0058; A61K 48/0075; C12N 15/86; C12N 2750/14132; C12N 2750/14143

(57) ABSTRACT

Provided herein are compositions, kits, and methods for assessing and monitoring disease stages and phases, predicting the likelihood of disease progression, predicting and monitoring responses to disease therapies, and treating disease conditions.

### CROSS-REFERENCE TO RELATED APPLICATIONS/CLAIM OF PRIORITY

The present application claims priority to U.S. Provisional Patent Application Ser. No. 63/490,144, filed, Mar. 14, 2023 and U.S. Provisional Patent Application Ser. No. 63/490,383, filed, Mar. 15, 2023, herein incorporated by reference in their entirety.

### SEQUENCE LISTING

The text of the computer readable sequence listing filed herewith, titled “STDU2-41804-601_SQL.xml”, created Mar. 14, 2024, having a file size of 6,652 bytes, is hereby incorporated by reference in its entirety.

### FIELD

Provided herein are compositions, kits, and methods for assessing and monitoring disease stages and phases, predicting the likelihood of disease progression, predicting and monitoring responses to disease therapies, and treating disease conditions.

### BACKGROUND

The blood-brain barrier (BBB) is vital for maintaining brain homeostasis by enabling control of exchange of compounds between the blood and the brain parenchyma. The BBB prevents unwanted toxins and pathogens from entering the brain. This barrier, however, breaks down with age. This breakdown is associated with many age-related disorders. Techniques for preventing, compensation for, or reversing this breakdown are needed.

### SUMMARY

In some embodiments, provided herein is a method for treating age-related, neurological, and vascular diseases or conditions comprising administering to a subject a brain endothelial specific vector (e.g., AAV) wherein the brain endothelial specific vector comprises a nucleic acid that encodes a mucin (e.g., a glycosylated protein) and/or a mucin associated protein (e.g., a membrane glycoprotein (e.g., a carbohydrate-active enzyme (e.g., a glycosyltransferase))).

In some embodiments, provided herein is a method for treating age-related, neurological, and vascular diseases or conditions comprising genetically modifying brain endothelial cells to express mucin (e.g., a glycosylated protein) and/or mucin associated proteins (e.g., membrane glycoproteins (e.g., carbohydrate-active enzyme (e.g., glycosyltransferases)) or extend mucin-type-O-glycosylation.

In some embodiments, provided herein is a method for treating age-related, neurological, and vascular diseases or conditions comprising adding a heterologous mucin (e.g., a glycosylated protein) and/or a mucin associated protein (e.g., a membrane glycoprotein (e.g., a carbohydrate-active enzyme (e.g., a glycosyltransferase))) to a brain endothelial cell. In some embodiments, the abundance and/or activity of the one or more mucin (e.g., a glycosylated protein) and/or a mucin associated protein (e.g., a membrane glycoprotein (e.g. a carbohydrate-active enzyme (e.g., a glycosyltransferase))) is achieved by administering the selected mucin and/or mucin associated protein, or an engineered variant thereof. In one implementation, the mucin and/or mucin associated protein, or an engineered variant thereof is combined with (e.g., conjugated to or packaged within) a targeting moiety which increases the abundance of the foregoing in the endothelial lumen of vessels in the brain and/or increases mucin-type-O-glycosylation on the lumen and/or the abluminal surfaces of endothelial cells in the brain.

In some embodiments, the method comprises any one or more of maintaining the integrity of the glycocalyx, reducing BBB permeability, reducing brain inflammation, reducing age-related cognitive decline, treating neurodegenerative disease by any one or more of increasing thickness/mass of the glycocalyx, increasing mucin content and/or mucin associated protein content, or an engineered variant thereof of the glycocalyx layer, and restoring normal mucin and/or mucin associated protein biosynthetic activity in the glycocalyx layer.

In some embodiments the methods find use in preventing, slowing, or reducing symptoms or pathologies associated with a disease or condition (e.g., a subject having, suspected of having, or at risk of Alzheimer's disease, Parkinson's disease, frontotemporal dementia, multiple sclerosis, traumatic brain injury, stroke, amyotrophic lateral sclerosis, Huntington's Disease, etc.).

In some embodiments, provided herein is a composition comprising a brain endothelial specific vector (e.g., AAV) wherein the brain endothelial specific vector comprises a nucleic acid that encodes a mucin associated protein (e.g., a membrane glycoprotein (e.g., a carbohydrate-active enzyme (e.g., a glycosyltransferase))).

In some embodiments, the mucin is a transmembrane mucin, (e.g., MUC1 (e.g., Gene ID: 4582), MUC4 (e.g., Gene ID: 4585), MUC 16 (e.g., Gene ID: 94025)), a secreted mucin (e.g., gel-forming (e.g., MUC2 (e.g., Gene ID: 4583), MUC5AC (e.g., Gene ID: 4586), MUC6 (e.g., Gene ID: 4588))) or a soluble mucin (e.g., non-gel-forming (e.g., MUC7 (e.g., Gene ID: 4589), MUC8 (e.g., Gene ID: 100129528), MUC9 (e.g., MUC5B (e.g., Gene ID: 727897)), MUC20 (e.g., Gene ID: 200958))).

In some embodiments, the mucin is a mucin domain-containing glycoprotein (e.g., PODXL (e.g. Gene ID: 5420), CD34 (e.g. Gene ID: 947), DAG1 (e.g. Gene ID: 1605), MCAM (e.g. Gene ID: 4162), NID1 (e.g. Gene ID: 4811), NID2 (e.g. Gene ID: 22795), AGRN (e.g. Gene ID: 375790), APP (e.g. Gene ID: 351), CD164 (e.g. Gene ID: 8763), EMCN (e.g. Gene ID: 51705), LRP8 (e.g. Gene ID: 7804), CD248 (e.g. Gene ID: 57124), PRG4 (e.g. Gene ID: 10216), CD55 (e.g. Gene ID: 1604), CD44 (e.g. Gene ID: 960), SDC3 (e.g. Gene ID: 9672), SDC1 (e.g. Gene ID: 6382), HEG1 (e.g. Gene ID: 57493), CCDC80 (e.g. Gene ID: 151887), GP1BA (e.g. Gene ID: 2811), VASN (e.g. Gene ID: 114990), LYVE1 (e.g. Gene ID: 10894), ABI3BP (e.g. Gene ID: 25890), F5 (e.g. Gene ID: 2153), PLXDC2 (e.g. Gene ID: 84898), FNDC1 (e.g. Gene ID: 84624), MEGF9 (e.g. Gene ID: 1955).

In some embodiments, in any of the preceding aspects, the mucin associated protein is B3GNT2 (e.g. Gene ID: 10678), B3GNT3 (e.g. Gene ID: 10331), B3GNT4 (e.g. Gene ID: 79369), B3GNT5 (e.g. Gene ID: 84002), B3GNT6 (e.g. Gene ID: 192134), B3GNT7 (e.g. Gene ID: 93010), B3GNT8 (e.g. Gene ID: 374907), B4GALT1 (e.g. Gene ID: 2683), B4GALT2 (e.g. Gene ID: 8704), B4GALT3 (e.g. Gene ID: 8703), B4GALT4 (e.g. Gene ID: 8702), B4GALT5 (e.g. Gene ID: 9334), B4GALT6 (e.g. Gene ID: 9331), B4GALT7 (e.g. Gene ID: 11285), C1GALT1 (e.g. Gene ID: 56913), GALNT1 (e.g. Gene ID: 2589), GALNT2 (e.g. Gene ID: 2590), GALNT3 (e.g. Gene ID: 2591), GALNT4 (e.g. Gene ID: 8693), GALNT5 (e.g. Gene ID: 11227), GALNT6 (e.g. Gene ID: 11226), GALNT7 (e.g. Gene ID: 51809), GALNT8 (e.g. Gene ID: 26290), GALNT9 (e.g. Gene ID: 50614), GALNT10 (e.g. Gene ID: 55568), GALNT11 (e.g. Gene ID: 63917), GALNT12 (e.g. Gene ID: 79695), GALNT13 (e.g. Gene ID: 114805), GALNT14 (e.g. Gene ID: 79623), GALNT15 (e.g. Gene ID: 117248), GALNT16 (e.g. Gene ID: 57452), GALNT17 (e.g. Gene ID: 64409), GALNT18 (e.g. Gene ID: 374378), GALNT19, GALNT20, ST3GAL1 (e.g. Gene ID: 6482), ST3GAL2 (e.g. Gene ID: 6843), ST3GAL4 (e.g. Gene ID: 6484), ST6GALNAC1 (e.g. Gene ID: 55808), ST6GALNAC3 (e.g. Gene ID: 256435), ST6GALNAC4 (e.g. Gene ID: 27090), or functional variants thereof.

In some embodiments, in any of the preceding aspects, the vector is a viral vector, a lipid nanoparticle, or a cationic particle.

In some embodiments, in any of the preceding aspects, the condition is one or more of Alzheimer's disease, Parkinson's disease, frontotemporal dementia, multiple sclerosis, traumatic brain injury, stroke, and/or amyotrophic lateral sclerosis.

In some embodiments, in any of the preceding aspects, the vector is co-administered with a targeting moiety.

In some embodiments, the condition is one or more of accumulation of genetic damage, reduced telomere length, epigenetic alterations in DNA, impairment of protein homeostasis, deregulated nutrient sensing, cellular senescence, stem cell exhaustion, mitochondrial dysfunction, altered cellular communication, neurodegenerative diseases, cardiovascular diseases, cerebral amyloid angiopathy, cerebrovascular diseases, cancer, autism spectrum disorder, central nervous system, immune system disorders, neurological/neurodegenerative diseases with BBB dysregulation, or musculoskeletal disorders.

### BRIEF DESCRIPTION OF THE DRAWINGS

FIGS. 1A-15B show the plasmids used for overexpression of B3GNT3 and C1GALT1. FIG. 1A shows the plasmids used for overexpression of C1GALT1. FIG. 1B shows the plasmids used for overexpression of B3GNT3.

FIG. 2A-20 show that the brain endothelial glycocalyx is highly dysregulated during aging. FIG. 2A Diagram of the blood-brain barrier (BBB) and brain endothelial glycocalyx layer. FIG. 2B Transmission electron micrographs of cortical capillaries with lanthanum nitrate staining from young (3-month-old) and aged (21-month-old) mice. Scale bar=1 μm. FIG. 2C Quantification of luminal endothelial glycocalyx heights of young (3-month-old) and aged (21-month-old) mice (n=4 mice per group; two-sided t-test; mean±s.e.m.). FIG. 2D Quantification of luminal endothelial glycocalyx areas of young (3-month-old) and aged (21-month-old) mice (n=4 mice per group; two-sided t-test; mean±s.e.m.). FIG. 2E Volcano plot of differentially expressed glycosylation-related genes (glyco-genes) in brain endothelial cells from young (3-month-old) and aged (19-month-old) mice (genes upregulated with age in red and genes downregulated with age in blue). Original bulk RNA-seq data from Yousef, et al. 2019. FIG. 2F Top glycosylation-related pathways upregulated and downregulated with age in brain endothelial cells. FIG. 2G Experimental scheme for glycan and glycoconjugate profiling of brain endothelial cells via microvessel (MV) imaging and flow cytometry. FIG. 2H Mucin-domain glycoprotein expression and Lycopersicon esculentum lectin (LEL; endothelial marker) labeling in acutely isolated microvessels. Scale bar=10 μm. FIG. 2I Quantification of FIG. 2H (n=4 mice per group; two-sided t-test; mean±s.e.m.). FIG. 2J shows median fluorescence intensity (MFI) of heparan sulfate (via 10E4 antibody). FIG. 2K shows median fluorescence intensity (MFI) of chondroitin sulfate (via CS-56 antibody). FIG. 2L shows median fluorescence intensity (MFI) of hyaluronan (via versican). FIG. 2M shows median fluorescence intensity (MFI) of mucin-domain glycoproteins (via StcE^{E447D}-AF647). FIG. 2N shows median fluorescence intensity (MFI) of x2,6-linked sialic acids (via SNA lectin). FIG. 2O shows median fluorescence intensity (MFI) of x2,3-linked sialic acids (via MAAII lectin). Each of 2J-2O shown on mechanically isolated brain endothelial cells from young (3-month-old) and aged (21-month-old) mice (n=7 mice per group; two-sided t-test; mean±s.e.m.).

FIGS. 3A-30 show that mucin-type O-glycosylation is downregulated in brain endothelial cells during aging and neurodegenerative disease. FIG. 3A shows a diagram of mucin binding and degrading tools used in experiments. FIG. 3B shows luminal mucin-domain glycoprotein expression based on StcE^{E447D}-AF647 labeling in CD31⁺ cortical vasculature. Scale bar=20 μm. FIG. 3C shows quantification of FIG. 3B (n=4 mice per group; two-sided t-test; mean±s.e.m.). FIG. 3D shows luminal sialic acid expression based on SNA-Cy3 labeling in CD31 cortical vasculature. Scale bar=20 μm. FIG. 3E shows quantification of FIG. 3D (n=3 mice per group; two-sided t-test; mean±s.e.m.). FIG. 3F shows transmission electron micrographs of cortical capillaries with lanthanum nitrate staining from mice treated with StcE^{E447D} or StcE for 24 h. Scale bar=1 μm. FIG. 3G shows quantification of luminal endothelial glycocalyx heights of mice treated with StcE^{E447D} or StcE (n=3 mice per group; two-sided t-test; mean±s.e.m.). FIG. 3H shows histograms of luminal endothelial glycocalyx heights including 5-6 vessels from each animal in FIG. 3G. Fitted gaussian distributions are displayed for each group. FIG. 3I shows quantification of luminal endothelial glycocalyx areas of mice treated with StcE^{E447D} or StcE (n=3 mice per group; two-sided t-test; mean±s.e.m.). FIG. 3J shows histograms of luminal endothelial glycocalyx areas including 5-6 vessels from each animal in FIG. 3I. Fitted gaussian distributions are displayed for each group. FIG. 3K shows C1GALT1 expression (red) in acutely isolated microvessels labeled with LEL. Scale bar=10 μm. FIG. 3L shows quantification of FIG. 3K (n=4, two-sided t-test; mean±s.e.m.). FIG. 3M shows B3GNT3 expression (red) in acutely isolated microvessels labeled with LEL. Scale bar=10 μm. FIG. 7N shows quantification of FIG. 3M (n=4, two-sided t-test; mean±s.e.m.). FIG. 3O shows mucin-type O-glycan biosynthetic pathway. Broad transcriptional downregulation of brain endothelial core 1 mucin-type O-glycan biosynthetic enzymes is observed in mouse aging, Alzheimer's disease, and Huntington's disease RNA-seq datasets.

FIGS. 4A-4M shows that loss or truncation of brain endothelial mucin-type 0-glycans leads to increased BBB leakiness and brain bleeding. FIG. 4A shows an overview of AAV constructs and C1galt1 knockdown paradigm. FIG. 4B shows C1GALT1 expression and mucin-domain glycoprotein labeling in acutely isolated microvessels. Scale bar-10 μm. FIG. 4C shows quantification of C1GALT1 expression in FIG. 4B (n=5, two-sided t-test; mean±s.e.m.). FIG. 4D shows quantification of mucin-domain glycoprotein labeling in FIG. 4B (n=5, two-sided t-test; mean±s.e.m.). FIG. 4E shows sulfo-NHS-biotin leakage in the cortices of young mice transduced with AAV-EGFP and AAV-miR-C1galt1. FIG. 4F shows sulfo-NHS-biotin leakage (indicated by white triangles) from EGFP⁺ cortical vessels of AAV-miR-C1GALT 1-transduced mice. Scale bar=50 μm. FIG. 4G shows quantification of cortical vessel permeability to sulfo-NHS-biotin in FIG. 4F (n=5 mice per group; two-sided t-test; mean±s.e.m.). FIG. 4H shows an overview of luminal mucin-domain glycoprotein degradation paradigm using StcE. FIG. 4I shows brains from StcE-treated mice exhibit signs of hemorrhaging. FIG. 4J shows H&E images of cerebral bleeds in the meninges and ventricles of StcE-treated mice. Scale bar=50 μm. FIG. 4K shows whole brain images of sulfo-NHS-biotin leakage in StcE-treated mice. Scale bar=1 mm. FIG. 4L Sulfo-NHS-biotin leakage (white) from cortical vessels (green) of StcE-treated mice. Scale bar=50 μm. FIG. 4M shows quantification of cortical vessel permeability to sulfo-NHS-biotin in FIG. 4L (n=4 mice per group; two-sided t-test; mean±s.e.m.).

FIGS. 5A-5M show that restoration of mucin-type O-glycosylation improves BBB function in aged mice. FIG. 5A shows an overview of C1GALT1 and B3GNT3 overexpression paradigm and relevant AAV constructs. FIG. 5B shows C1GALT1 expression in acutely isolated microvessels labeled with LEL. Scale bar=10 μm. FIG. 5C shows quantification of FIG. 5B (n=5 mice per group; two-sided t-test; mean±s.e.m.). FIG. 5D shows B3GNT3 expression in acutely isolated microvessels labeled with LEL. Scale bar=10 μm. FIG. 5E shows quantification of FIG. 5D (n=5 mice per group; two-sided t-test; mean±s.e.m.). FIG. 5F shows quantification of mucin-domain glycoprotein labeling in acutely isolated microvessels (n=5 mice per group; two-sided t-test; mean±s.e.m.). FIG. 5G shows sulfo-NHS-biotin leakage in whole brain sections of AAV-transduced mice. FIG. 5H shows sulfo-NHS-biotin leakage (indicated by white triangles) from EGFP⁺ cortical vessels of AAV-transduced mice. Scale bar=50 μm. FIG. 5I shows quantification of FIG. 5H (n=5 mice per group; two-sided t-test; mean±s.e.m.). FIG. 5J shows iba1 (cyan) and CD68 (red) expression in the cortices of AAV-transduced mice. Scale bar=50 μm. FIG. 5K shows quantification of CD68⁺ signal in cortical Iba1 microglia in FIG. 5J (n=5 mice per group; two-sided t-test; mean±s.e.m.). FIG. 5L shows quantification of cortical Iba1⁺ area in FIG. 5J (n=5 mice per group; two-sided t-test; mean±s.e.m.). FIG. 5M shows a schematic of BBB dysfunction during aging and neurodegenerative disease. The brain endothelial glycocalyx layer degenerates with aging which contributes to dysregulated transport across the BBB.

FIGS. 6A-6D show heterogeneity in the brain endothelial glycocalyx layer. FIG. 6A shows a diagram of glycocalyx height and area measurements. FIG. 6B shows transmission electron micrographs of cortical capillaries with lanthanum nitrate staining from young (3-month-old) and aged (21-month-old) mice. Representative images of capillaries with glycocalyx measurements approximately in the high (80^{th}-90^{th} percentiles), medium (45^{th}-55^{th} percentiles), and low (10^{th}-20^{th} percentiles) ranges of all vessels captured for each group were chosen. Scale bar=1 μm. FIG. 6C shows histograms of luminal endothelial glycocalyx heights including 5-6 vessels from each animal. Fitted gaussian distributions are displayed for each group. FIG. 6D shows histograms of luminal endothelial glycocalyx areas including 5-6 vessels from each animal. Fitted gaussian distributions are displayed for each group

FIGS. 7A-7J show compositional changes in the brain endothelial glycocalyx during aging. FIG. 7A shows flow cytometry experimental scheme comparing mechanical (blue) and enzymatic (orange) brain dissociation methods demonstrating that mechanical dissociation better preserves several classes of glycans/glycoconjugates on brain endothelial cells. FIG. 7B shows hyaluronan, heparan sulfate, and α2,6-linked sialic acid expression in acutely isolated microvessels from young (3-month-old) and aged (21-month-old) mice. Scale bar=10 μm. FIG. 7C shows quantification of hyaluronan in FIG. 7B (n=4, two-sided t-test; mean±s.e.m.). FIG. 7D shows quantification of heparan sulfate in FIG. 7B (n=4, two-sided t-test; mean±s.e.m.). FIG. 7E shows quantification of α2,6-linked sialic acid in FIG. 7B (n=4, two-sided t-test; mean±s.e.m.). FIG. 7F shows α2,3-linked sialic acid, chondroitin sulfate, and terminal GalNAc expression in acutely isolated microvessels from young (3-month-old) and aged (21-month-old) mice. Scale bar=10 μm. FIG. 7G shows quantification of α2,3-linked sialic acid in FIG. 7F (n=4, two-sided t-test; mean±s.e.m.). FIG. 7H shows quantification of chondroitin sulfate in FIG. 7F (n=4, two-sided t-test; mean±s.e.m.). FIG. 7I shows quantification of terminal GalNAc in FIG. 7F (n=4, two-sided t-test; mean±s.e.m.). FIG. 7J is a summary of aging-associated changes in brain endothelial glycan/glycoconjugate classes observed using bulk RNA-seq, fluorescence microvessel imaging, and flow cytometry profiling.

FIGS. 8A-8D show luminal mucin domain labeling via StcE^{E447D}-AF647 is not significantly changed in the heart or liver with age. FIG. 8A shows luminal mucin-domain glycoprotein expression in CD31⁺ vasculature in the hearts of young (3-month-old) and aged (21-month-old) mice. Scale bar=20 μm. FIG. 8B shows quantification of FIG. 8A (n=3 mice per group; two-sided t-test; mean±s.e.m.). FIG. 8C shows luminal mucin-domain glycoprotein expression in CD31⁺ vasculature in the livers of young (3-month-old) and aged (21-month-old) mice. Scale bar=20 μm. FIG. 8D shows quantification of FIG. 8C (n=3 mice per group; two-sided t-test; mean±s.e.m.).

FIGS. 9A-9F show luminal cerebrovascular proteomics does not reveal substantial downregulation of mucin-domain glycoprotein scaffolds with age. FIG. 9A shows luminal cerebrovascular proteomics scheme. FIG. 9B shows a volcano plot of luminal cerebrovascular proteins identified in young (3-month-old) versus aged (21-month-old) mice (n=6 mice per group). Notable mucin-domain glycoproteins are highlighted in purple. Proteins only detected in young animals (in ≥50% of young animals) are highlighted in blue in the upper lefthand corner. Proteins only detected in aged animals (in ≥50% of aged animals) are highlighted in red in the upper righthand corner. FIG. 9C shows principal component analysis (PCA) of samples in FIG. 9B (n=6 mice per group). FIG. 9D shows gene ontology (GO) term cellular component and biological process analysis of all proteins detected in luminal cerebrovascular proteomics experiment. FIG. 9E shows NID1 expression in cortical vessels does not correlate with StcE^{E447D}-AF647 signal as indicated by the white triangles. Scale bar=50 μm. FIG. 9F shows quantification of NID1 expression in COL4A⁺ vasculature in FIG. 9E (n=3 mice per group; two-sided t-test; mean±s.e.m.).

FIGS. 10A-10F show transcriptional dysregulation in brain endothelial glycosylation pathways in neurodegenerative diseases. FIG. 10A shows a volcano plot of differentially expressed glyco-genes in brain endothelial cells from Alzheimer's disease (AD) patients and age-matched controls (genes upregulated with AD in red and genes downregulated with AD in blue). Original snRNA-seq data from Yang, et al. 2022. FIG. 10B shows upregulated and downregulated glycosylation-related pathways in brain endothelial cells in AD patients versus age-matched controls. FIG. 10C shows a volcano plot of differentially expressed glyco-genes in brain endothelial cells from Huntington's disease (HD) patients and age-matched controls (genes upregulated with HD in red and genes downregulated with HD in blue). FIG. 10D shows upregulated and downregulated glycosylation-related pathways in brain endothelial cells in HD patients versus age-matched controls. FIG. 10E shows a venn diagram of significantly upregulated brain endothelial glyco-genes that are shared amongst mouse aging, AD, and HD datasets. FIG. 10F shows a venn diagram of significantly downregulated brain endothelial glyco-genes that are shared amongst mouse aging, AD, and HD datasets. Mucin-type O-glycan biosynthetic enzymes are highlighted in purple.

FIGS. 11A-11B show modulation of mucin-type O-glycosylation in bEnd.3 cells. FIG. 11A shows reduction of StcE^{E447D}-AF647 binding following knockdown of C1galt1 in bEnd.3 cells using AAV-miR-E constructs compared to AAV-EGFP (n=4 per group; two-sided t-test; mean±s.e.m.). FIG. 11B shows increased StcE^{E447D}-AF647 binding following overexpression of C1GALT and B3GNT3 in bEnd.3 cells using AAV-C1GALT1 and AAV-B3GNT3 compared to AAV-EGFP (n=4 per group; two-sided t-test; mean±s.e.m.).

FIGS. 12A-12C show that intravenous StcE treatment impairs BBB integrity. FIG. 12A shows additional brains from StcE-treated mice exhibiting hemorrhaging (n=4 per group). FIG. 12B shows sulfo-NHS-biotin leakage (white) from blood vessels in different brain regions of StcE-treated mice. Scale bar=50 μm. FIG. 12C shows quantification of sulfo-NHS-biotin permeability in different brain regions in saline- and StcE-treated mice (n=4 mice per group; two-sided t-test; mean±s.e.m.).

FIGS. 13A-13B show that restoration of mucin-type O-glycosylation improves spatial working memory in aged mice. FIG. 13A shows a schematic of administration of AAV constructs to aged (16-month-old) and young (3-month-old) mice and cognitive testing timeline. FIG. 13B shows how spatial working memory was assessed using the Y Maze as percent spontaneous alternations (n=26 (Aged EGFP), 19 (Aged C1GALT1), 23 (Aged B3GNT3), and 20 (Young EGFP); two-sided t-test; mean±s.e.m.).

### DETAILED DESCRIPTION

Provided herein are compositions and methods for assessing and monitoring disease stages and phases, predicting the likelihood of disease progression, predicting and monitoring responses to disease therapies, and treating disease conditions.

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures.

The singular forms “a,” “and” and “the” include plural references unless the context clearly dictates otherwise.

The present disclosure also contemplates other embodiments “comprising,” “consisting of” and “consisting essentially of,” the embodiments or elements presented herein, whether explicitly set forth or not.

Ranges can be expressed herein as from “about” one particular value, and/or to “about” another particular value. When such a range is expressed, some embodiments includes from the one particular value and/or to the other particular value. Similarly, when values are expressed as approximations, by use of the antecedent “about,” it will be understood that the particular value forms an embodiment. It will be further understood that the endpoints of each of the ranges are significant both in relation to the other endpoint, and independently of the other endpoint. It is also understood that there are a number of values disclosed herein, and that each value is also herein disclosed as “about” that particular value disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed, then “less than or equal to 10” as well as “greater than or equal to 10” are also disclosed.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

As used herein, the term “adeno-associated virus” (AAV), includes but is not limited to, AAV type 1, AAV type 2, AAV type 3 (including types 3A and 3B), AAV type 4, AAV type 5, AAV type 6, AAV type 7, AAV type 8, AAV type 9, AAV type 10, AAV type 11, AAV type 12, AAV type 13, AAV type rh32.33, AAV type rh8, AAV type rh10, AAV type rh74, AAV type hu.68, avian AAV, bovine AAV, canine AAV, equine AAV, ovine AAV, snake AAV, bearded dragon AAV, AAV2i8, AAV2g9, AAV-LK03, AAV7m8, AAV Anc80, AAV PHP.B, and any other AAV including chimeric AAV. See, e.g., BERNARD N. FIELDS et al., VIROLOGY volume 2, chapter 69 (4th ed., Lippincott-Raven Publishers). A number of AAV serotypes and clades have been identified (see, e.g., Gao et al, (2004) J. Virology 78:6381-6388; Moris et al, (2004) Virology 33-:375-383). “Adeno-associated virus” or AAV also encompasses chimeric AAV. The term “chimeric AAV” refers to an AAV comprising a protein capsid comprising capsid protein subunits with regions, domains, individual amino acids that are derived from two or more different serotypes of AAV or another virus, including for example, another parvovirus.

The term “contacting” as used herein refers to bring or put in contact, to be in or come into contact. The term “contact” as used herein refers to a state or condition of touching or of immediate or local proximity. Contacting a composition to a target destination, such as, but not limited to, an organ, tissue, cell, or tumor, may occur by any means of administration known to the skilled artisan.

The terms “non-naturally occurring,” “engineered,” and “synthetic” are used interchangeably and indicate the involvement of the hand of man. The terms, when referring to nucleic acid molecules or polypeptides mean that the nucleic acid molecule or the polypeptide is at least substantially free from at least one other component with which they are naturally associated in nature and as found in nature.

As used herein, a “nucleic acid” or a “nucleic acid sequence” refers to a polymer or oligomer of pyrimidine and/or purine bases, preferably cytosine, thymine, and uracil, and adenine and guanine, respectively (See Albert L. Lehninger, Principles of Biochemistry, at 793800 (Worth Pub. 1982)). The present technology contemplates any deoxyribonucleotide, ribonucleotide, or peptide nucleic acid component, and any chemical variants thereof, such as methylated, hydroxymethylated, or glycosylated forms of these bases, and the like. The polymers or oligomers may be heterogenous or homogenous in composition and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states. In some embodiments, a nucleic acid or nucleic acid sequence comprises other kinds of nucleic acid structures such as, for instance, a DNA/RNA helix, peptide nucleic acid (PNA), morpholino nucleic acid (see, e.g., Braasch and Corey, Biochemistry, 41(14): 4503-4510 (2002)) and U.S. Pat. No. 5,034,506), locked nucleic acid (LNA; see Wahlestedt et al., Proc. Natl. Acad. Sci. U.S.A., 97: 5633-5638 (2000)), cyclohexenyl nucleic acids (see Wang, J. Am. Chem. Soc., 122: 8595-8602 (2000)), and/or a ribozyme. Hence, the term “nucleic acid” or “nucleic acid sequence” may also encompass a chain comprising non-natural nucleotides, modified nucleotides, and/or non-nucleotide building blocks that can exhibit the same function as natural nucleotides (e.g., “nucleotide analogs”); further, the term “nucleic acid sequence” as used herein refers to an oligonucleotide, nucleotide or polynucleotide, and fragments or portions thereof, and to DNA or RNA of genomic or synthetic origin, which may be single or double-stranded, and represent the sense or antisense strand. The terms “nucleic acid,” “polynucleotide,” “nucleotide sequence” are used interchangeably. They refer to a polymeric form of nucleotides of any length, either deoxyribonucleotides or ribonucleotides, or analogs thereof. The term “oligonucleotide,” or “oligos,” as used herein, generally refers to a short nucleic acid sequence comprising from about 2 to about 100 nucleotides (e.g., about 5, 10, 15, 20, 25, 30, 35, 40, 45, 50, 55, 60, 65, 70, 75, 80, 85, 90, 95, 99, or 100 nucleotides, or a range defined by any of the foregoing values) Any of the oligonucleotide sequences described herein may comprise, consist essentially of, or consist of a complement of any of the sequences disclosed herein.

The terms “protein,” “peptide,” and “polypeptide” are used interchangeably herein, and refer to a polymer of amino acid residues linked together by peptide bonds. The terms refer to a protein, peptide, or polypeptide of any size, structure, or function. Typically, a protein, peptide, or polypeptide will be at least three amino acids long. A protein, peptide, or polypeptide may refer to an individual protein or a collection of proteins. One or more of the amino acids in a protein, peptide, or polypeptide may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification, etc. A protein, peptide, or polypeptide may also be a single molecule or may be a multi-molecular complex. A protein, peptide, or polypeptide may be just a fragment of a naturally occurring protein or peptide. A protein, peptide, or polypeptide may be naturally occurring, engineered, or synthetic, or any combination thereof. Any of the proteins provided herein may be produced by any method known in the art. For example, the proteins provided herein may be produced via recombinant protein expression and purification, which is especially suited for fusion proteins comprising a peptide linker. Methods for recombinant protein expression and purification are well known, and include those described by Green and Sambrook, Molecular Cloning: A Laboratory Manual (4th ed., Cold Spring Harbor Laboratory Press, Cold Spring Harbor, N.Y. (2012)), the entire contents of which are incorporated herein by reference.

A “subject” or “patient” may be human or non-human and may include, for example, animal strains or species used as “model systems” for research purposes, such a mouse model as described herein. Likewise, patient may include either adults or juveniles (e.g., children). Moreover, patient may mean any living organism, preferably a mammal (e.g., human or non-human) that may benefit from the administration of proteins, nucleic acids, or compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish, and the like. In one embodiment of the methods provided herein, the mammal is a human.

As used herein, the terms “providing,” “administering,” and “introducing,” are used interchangeably herein and refer to the placement into a cell, organism, or subject by a method or route which results in at least partial localization to a desired site. For example, the compositions disclosed herein can be administered by any appropriate route which results in delivery to a desired location in the cell, organism, or subject.

A “vector” is a delivery system for a biological agent. For example, a vector may be used to deliver nucleic acid (e.g., DNA) to a cell. In some embodiments, the vector is itself nucleic acid (e.g., a viral vector, plasmid, or the like). In some embodiments, the vector is an “expression vector,” which is a replicon, such as plasmid, phage, virus, or cosmid, to which another DNA segment may be attached or incorporated so as to bring about the replication, transcription, or expression of the attached segment in a cell.

A cell has been “genetically modified,” “transformed,” or “transfected” by exogenous DNA, e.g., a recombinant expression vector, when such DNA has been introduced inside the cell. The presence of the exogenous DNA results in permanent or transient genetic change. The transforming DNA may or may not be integrated (covalently linked) into the genome of the cell. For example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones that comprise a population of daughter cells containing the transforming DNA. A “clone” is a population of cells derived from a single cell or common ancestor by mitosis. A “cell line” is a clone of a primary cell that is capable of stable growth in vitro for many generations.

Viral and non-viral based gene transfer methods can be used to introduce nucleic acids encoding components of the present system into cells, tissues, or a subject. Such methods can be used to administer nucleic acids encoding components of the present system to cells in culture, or in a host organism. Non-viral vector delivery systems include DNA plasmids, cosmids, RNA (e.g., a transcript of a vector described herein), a nucleic acid, and a nucleic acid complexed with a delivery vehicle. Viral vector delivery systems include DNA and RNA viruses, which have either episomal or integrated genomes after delivery to the cell. Viral vectors include, for example, retroviral, lentiviral, adenoviral, adeno-associated and herpes simplex viral vectors.

In certain embodiments, plasmids that are non-replicative, or plasmids that can be cured by high temperature may be used, such that any or all of the necessary components of the system may be removed from the cells under certain conditions.

A variety of viral constructs may be used to deliver the present system. Nonlimiting examples of such recombinant viruses include recombinant adeno-associated virus (AAV), recombinant adenoviruses, recombinant lentiviruses, recombinant retroviruses, recombinant herpes simplex viruses, recombinant poxviruses, phages, etc. The present disclosure provides vectors capable of integration in the host genome, such as retrovirus or lentivirus. See, e.g., Ausubel et al., Current Protocols in Molecular Biology, John Wiley & Sons, New York, 1989; Kay, M. A., et al., 2001 Nat. Medic. 7(1):33-40; and Walther W. and Stein U., 2000 Drugs, 60(2): 249-71, incorporated herein by reference.

To construct cells that express the present system, expression vectors for stable or transient expression of the present system may be constructed via conventional methods as described herein and introduced into host cells. For example, nucleic acids encoding the components of the present system may be cloned into a suitable expression vector, such as a plasmid or a viral vector in operable linkage to a suitable promoter. The selection of expression vectors/plasmids/viral vectors should be suitable for integration and replication in eukaryotic cells.

The vectors of the present disclosure may direct expression of the nucleic acid in a particular cell type (e.g., tissue-specific regulatory elements are used to express the nucleic acid). Such regulatory elements include promoters that may be tissue specific or cell specific. The term “tissue specific” as it applies to a promoter refers to a promoter that is capable of directing selective expression of a nucleotide sequence of interest to a specific type of tissue (e.g., seeds) in the relative absence of expression of the same nucleotide sequence of interest in a different type of tissue. The term “cell type specific” as applied to a promoter refers to a promoter that is capable of directing selective expression of a nucleotide sequence of interest in a specific type of cell in the relative absence of expression of the same nucleotide sequence of interest in a different type of cell within the same tissue. The term “cell type specific” when applied to a promoter also means a promoter capable of promoting selective expression of a nucleotide sequence of interest in a region within a single tissue.

As used herein, comprising a certain sequence or a certain SEQ ID NO usually implies that at least one copy of said sequence is present in recited peptide or polynucleotide. However, two or more copies are also contemplated.

### 2. COMPOSITIONS

Embodiments of the present disclosure include a composition comprising a brain endothelial specific vector (e.g., AAV) wherein the brain endothelial specific vector comprises a nucleic acid that encodes a mucin (e.g., a glycosylated protein) and/or a mucin associated protein (e.g., a membrane glycoprotein (e.g., a carbohydrate-active enzyme (e.g., a glycosyltransferase))).

In some embodiments, the composition comprises a mucin and/or a mucin associated protein, or an engineered variant thereof. In one implementation, mucin and/or mucin associated protein, or an engineered variant thereof is combined with (e.g., conjugated to or packaged within) a targeting moiety which increases the abundance of the foregoing in the endothelial lumen of vessels in the brain and/or increases mucin-type-O-glycosylation on the lumen and/or the abluminal surfaces of endothelial cells in the brain.

In some embodiments, the mucin is a transmembrane mucin, (e.g., MUC1 (e.g., Gene ID: 4582), MUC4 (e.g., Gene ID: 4585), MUC 16 (e.g., Gene ID: 94025)), a secreted mucin (e.g., gel-forming (e.g., MUC2 (e.g., Gene ID: 4583), MUC5AC (e.g., Gene ID: 4586), MUC6 (e.g., Gene ID: 4588))) or a soluble mucin (e.g., non-gel-forming (e.g., MUC7 (e.g., Gene ID: 4589), MUC8 (e.g., Gene ID: 100129528), MUC9 (e.g., MUC5B (e.g., Gene ID: 727897)), MUC20 (e.g., Gene ID: 200958))).

In some embodiments, the mucin is a mucin domain-containing glycoprotein (e.g., PODXL (e.g. Gene ID: 5420), CD34 (e.g. Gene ID: 947), DAG1 (e.g. Gene ID: 1605), MCAM (e.g. Gene ID: 4162), NID1 (e.g. Gene ID: 4811), NID2 (e.g. Gene ID: 22795), AGRN (e.g. Gene ID: 375790), APP (e.g. Gene ID: 351), CD164 (e.g. Gene ID: 8763), EMCN (e.g. Gene ID: 51705), LRP8 (e.g. Gene ID: 7804), CD248 (e.g. Gene ID: 57124), PRG4 (e.g. Gene ID: 10216), CD55 (e.g. Gene ID: 1604), CD44 (e.g. Gene ID: 960), SDC3 (e.g. Gene ID: 9672), SDC1 (e.g. Gene ID: 6382), HEG1 (e.g. Gene ID: 57493), CCDC80 (e.g. Gene ID: 151887), GP1BA (e.g. Gene ID: 2811), VASN (e.g. Gene ID: 114990), LYVE1 (e.g. Gene ID: 10894), ABI3BP (e.g. Gene ID: 25890), F5 (e.g. Gene ID: 2153), PLXDC2 (e.g. Gene ID: 84898), FNDC1 (e.g. Gene ID: 84624), MEGF9 (e.g. Gene ID: 1955).

In some embodiments, in any of the preceding aspects, the mucin associated protein is B3GNT2 (e.g. Gene ID: 10678), B3GNT3 (e.g. Gene ID: 10331), B3GNT4 (e.g. Gene ID: 79369), B3GNT5 (e.g. Gene ID: 84002), B3GNT6 (e.g. Gene ID: 192134), B3GNT7 (e.g. Gene ID: 93010), B3GNT8 (e.g. Gene ID: 374907), B4GALT1 (e.g. Gene ID: 2683), B4GALT2 (e.g. Gene ID: 8704), B4GALT3 (e.g. Gene ID: 8703), B4GALT4 (e.g. Gene ID: 8702), B4GALT5 (e.g. Gene ID: 9334), B4GALT6 (e.g. Gene ID: 9331), B4GALT7 (e.g. Gene ID: 11285), C1GALT1 (e.g. Gene ID: 56913), GALNT1 (e.g. Gene ID: 2589), GALNT2 (e.g. Gene ID: 2590), GALNT3 (e.g. Gene ID: 2591), GALNT4 (e.g. Gene ID: 8693), GALNT5 (e.g. Gene ID: 11227), GALNT6 (e.g. Gene ID: 11226), GALNT7 (e.g. Gene ID: 51809), GALNT8 (e.g. Gene ID: 26290), GALNT9 (e.g. Gene ID: 50614), GALNT10 (e.g. Gene ID: 55568), GALNT11 (e.g. Gene ID: 63917), GALNT12 (e.g. Gene ID: 79695), GALNT13 (e.g. Gene ID: 114805), GALNT14 (e.g. Gene ID: 79623), GALNT15 (e.g. Gene ID: 117248), GALNT16 (e.g. Gene ID: 57452), GALNT17 (e.g. Gene ID: 64409), GALNT18 (e.g. Gene ID: 374378), GALNT19, GALNT20, ST3GAL1 (e.g. Gene ID: 6482), ST3GAL2 (e.g. Gene ID: 6843), ST3GAL4 (e.g. Gene ID: 6484), ST6GALNAC1 (e.g. Gene ID: 55808), ST6GALNAC3 (e.g. Gene ID: 256435), ST6GALNAC4 (e.g. Gene ID: 27090), or functional variants thereof.

In some embodiments, the vector is a viral vector, a lipid nanoparticle, or a cationic particle.

In some embodiments, the vector is co-administered with a targeting moiety.

In some embodiments, the targeting (e.g., active (e.g., taking advantage of ligand-receptor, antigen-antibody, and other forms of molecular recognition) or passive targeting (e.g., taking advantage of abnormal tumor physiology and structure that results in the enhanced permeability and retention (EPR) effect)) moiety (e.g., a nanoparticle (e.g., a liposome, a micelle, a dendrimer, a nanosphere, or a nanocapsule)) is a peptide.

In some embodiments, the targeting moiety is a folate, is a transferrin, is an aptamer, is an antibody (e.g., a monoclonal antibody), or is an antibody fragment.

### 3. METHODS

Embodiments of the present disclosure also include a method for treating conditions and disorders comprising administering to a subject a brain endothelial specific vector (e.g., AAV) wherein the brain endothelial specific vector is a mucin (e.g., a glycosylated protein) and/or a mucin associated protein (e.g., a membrane glycoprotein e.g., a carbohydrate-active enzyme (e.g., a glycosyltransferase))).

In some embodiments, treating conditions and disorders include methods for treating age-related, neurological, and vascular diseases or conditions (e.g., accumulation of genetic damage, reduced telomere length, epigenetic alterations in DNA (e.g., modifications of chromatin (e.g., alterations in DNA methylation, histone modifications, and chromatin remodeling)), impairment of protein homeostasis (e.g., protein aggregation, post-translational modification, altered protein turnover (e.g., degradation of autophagy and the ubiquitin-proteasome system)), deregulated nutrient sensing (e.g., Nutrient sensing through insulin/IGF-1 (IIS)), cellular senescence, stem cell exhaustion, mitochondrial dysfunction, altered cellular communication, neurodegenerative diseases (e.g., Alzheimer's disease (AD), Parkinson's disease (PD), Frontotemporal dementia (FTD), Amyotrophic lateral sclerosis (ALS), Huntington's disease, cardiovascular diseases (CVD) (e.g., atherosclerosis, hypertension, myocardial infarction, and stroke), cerebral amyloid angiopathy (CAA), multiple sclerosis, traumatic brain injury (TBI), cerebrovascular diseases (e.g., epilepsy, stroke, carotid stenosis, vertebral stenosis, intracranial stenosis, aneurysms, and vascular malformations), cancer, autism spectrum disorder (e.g., complex neurodevelopment disorders caused by differences in the brain that affect communication and behavior), central nervous system (CNS) bacterial and viral infections (e.g., bacterial meningitis, (meningo)encephalitis, brain abscess, epidural abscess, and subdural empyema), immune system disorders (e.g., a shift in T-cell phenotype from native to memory, fewer early progenitor B cells, an increased proportion of mature T-cells, chronic low-grade inflammation), neurological/neurodegenerative diseases with BBB dysregulation, musculoskeletal disorders (sarcopenia (e.g., the loss of muscle mass and function) and osteoarthritis (OA)), etc.).

In some embodiments, the abundance and/or activity of the one or more mucin (e.g., a glycosylated protein) and/or a mucin associated protein (e.g., a membrane glycoprotein (e.g., a carbohydrate-active enzyme (e.g., a glycosyltransferase))) is achieved by administering to a subject the selected mucin and/or mucin associated protein, or an engineered variant thereof. In one implementation, the mucin and/or mucin associated protein, or an engineered variant thereof is combined with (e.g., conjugated to or packaged within) a targeting moiety which increases the abundance of the foregoing in the endothelial lumen of vessels in the brain and/or increases mucin-type-O-glycosylation on the lumen and/or the abluminal surfaces of endothelial cells in the brain.

Embodiments of the present disclosure also include a method for treating age-related, neurological, and vascular diseases or conditions comprising genetically modifying brain endothelial cells to express a mucin and/or a mucin associated protein (e.g., a membrane glycoprotein (e.g., a carbohydrate-active enzyme (e.g., a glycosyltransferase))).

Embodiments of the present disclosure also include a method for treating age-related, neurological, and vascular diseases or conditions comprising adding a heterologous mucin and/or mucin associated protein (e.g., a membrane glycoprotein (e.g., a carbohydrate-active enzyme (e.g., a glycosyltransferase))) to a brain endothelial cell.

In some embodiments, the methods comprise any one or more of maintaining the integrity of the glycocalyx, reducing BBB permeability, reducing brain inflammation, reducing age-related cognitive decline,

In some embodiments, the methods for treating neurodegenerative disease comprise any one or more of increasing thickness/mass of the glycocalyx, increasing the mucin content and/or mucin associated protein content of the glycocalyx layer, and restoring normal mucin biosynthetic activity in the glycocalyx layer.

In some embodiments, the methods find use in preventing, slowing, or reducing symptoms or pathologies associated with a disease or condition (e.g., a subject having, suspected of having, or at risk of age-related, neurological, and vascular diseases or conditions (e.g., accumulation of genetic damage, reduced telomere length, epigenetic alterations in DNA (e.g., modifications of chromatin (e.g., alterations in DNA methylation, histone modifications, and chromatin remodeling)), impairment of protein homeostasis (e.g., protein aggregation, post-translational modification, altered protein turnover (e.g., degradation of autophagy and the ubiquitin-proteasome system)), deregulated nutrient sensing (e.g., Nutrient sensing through insulin/IGF-1 (IIS)), cellular senescence, stem cell exhaustion, mitochondrial dysfunction, altered cellular communication, neurodegenerative diseases (e.g., Alzheimer's disease (AD), Parkinson's disease (PD), Frontotemporal dementia (FTD), Amyotrophic lateral sclerosis (ALS), Huntington's disease, cardiovascular diseases (CVD) (e.g., atherosclerosis, hypertension, myocardial infarction, and stroke), cerebral amyloid angiopathy (CAA), multiple sclerosis, traumatic brain injury (TBI), cerebrovascular diseases (e.g., epilepsy, stroke, carotid stenosis, vertebral stenosis, intracranial stenosis, aneurysms, and vascular malformations), cancer, autism spectrum disorder (e.g., complex neurodevelopment disorders caused by differences in the brain that affect communication and behavior), central nervous system (CNS) bacterial and viral infections (e.g., bacterial meningitis, (meningo)encephalitis, brain abscess, epidural abscess, and subdural empyema), immune system disorders (e.g., a shift in T-cell phenotype from native to memory, fewer early progenitor B cells, an increased proportion of mature T-cells, chronic low-grade inflammation), neurological/neurodegenerative diseases with BBB dysregulation, musculoskeletal disorders (sarcopenia (e.g., the loss of muscle mass and function) and osteoarthritis (OA)), etc.).

In some embodiments, the methods find use in preventing, slowing, or reducing symptoms or pathologies associated with and/or mechanisms caused by BBB breakdown.

In some embodiments, the methods find use in preventing, slowing, or reducing symptoms or pathologies associated with and/or mechanisms caused by BBB permeability.

In some embodiments, the mucin is a transmembrane mucin, (e.g., MUC1 (e.g., Gene ID: 4582), MUC4 (e.g., Gene ID: 4585), MUC 16 (e.g., Gene ID: 94025)), a secreted mucin (e.g., gel-forming (e.g., MUC2 (e.g., Gene ID: 4583), MUC5AC (e.g., Gene ID: 4586), MUC6 (e.g., Gene ID: 4588))) or a soluble mucin (e.g., non-gel-forming (e.g., MUC7 (e.g., Gene ID: 4589), MUC8 (e.g., Gene ID: 100129528), MUC9 (e.g., MUC5B (e.g., Gene ID: 727897)), MUC20 (e.g., Gene ID: 200958))).

In some embodiments, the mucin is a mucin domain-containing glycoprotein (e.g., PODXL (e.g. Gene ID: 5420), CD34 (e.g. Gene ID: 947), DAG1 (e.g. Gene ID: 1605), MCAM (e.g. Gene ID: 4162), NID1 (e.g. Gene ID: 4811), NID2 (e.g. Gene ID: 22795), AGRN (e.g. Gene ID: 375790), APP (e.g. Gene ID: 351), CD164 (e.g. Gene ID: 8763), EMCN (e.g. Gene ID: 51705), LRP8 (e.g. Gene ID: 7804), CD248 (e.g. Gene ID: 57124), PRG4 (e.g. Gene ID: 10216), CD55 (e.g. Gene ID: 1604), CD44 (e.g. Gene ID: 960), SDC3 (e.g. Gene ID: 9672), SDC1 (e.g. Gene ID: 6382), HEG1 (e.g. Gene ID: 57493), CCDC80 (e.g. Gene ID: 151887), GPIBA (e.g. Gene ID: 2811), VASN (e.g. Gene ID: 114990), LYVE1 (e.g. Gene ID: 10894), ABI3BP (e.g. Gene ID: 25890), F5 (e.g. Gene ID: 2153), PLXDC2 (e.g. Gene ID: 84898), FNDC1 (e.g. Gene ID: 84624), MEGF9 (e.g. Gene ID: 1955).

In some embodiments, in any of the preceding aspects, the mucin associated protein is B3GNT2 e.g. Gene ID: 10678), B3GNT3 (e.g. Gene ID: 10331), B3GNT4 (e.g. Gene ID: 79369), B3GNT5 (e.g. Gene ID: 84002), B3GNT6 (e.g. Gene ID: 192134), B3GNT7 (e.g. Gene ID: 93010), B3GNT8 (e.g. Gene ID: 374907), B4GALT1 (e.g. Gene ID: 2683), B4GALT2 (e.g. Gene ID: 8704), B4GALT3 (e.g. Gene ID: 8703), B4GALT4 (e.g. Gene ID: 8702), B4GALT5 (e.g. Gene ID: 9334), B4GALT6 (e.g. Gene ID: 9331), B4GALT7 (e.g. Gene ID: 11285), C1GALT1 (e.g. Gene ID: 56913), GALNT1 (e.g. Gene ID: 2589), GALNT2 (e.g. Gene ID: 2590), GALNT3 (e.g. Gene ID: 2591), GALNT4 (e.g. Gene ID: 8693), GALNT5 (e.g. Gene ID: 11227), GALNT6 (e.g. Gene ID: 11226), GALNT7 (e.g. Gene ID: 51809), GALNT8 (e.g. Gene ID: 26290), GALNT9 (e.g. Gene ID: 50614), GALNT10 (e.g. Gene ID: 55568), GALNT11 (e.g. Gene ID: 63917), GALNT12 (e.g. Gene ID: 79695), GALNT13 (e.g. Gene ID: 114805), GALNT14 (e.g. Gene ID: 79623), GALNT15 (e.g. Gene ID: 117248), GALNT16 (e.g. Gene ID: 57452), GALNT17 (e.g. Gene ID: 64409), GALNT18 (e.g. Gene ID: 374378), GALNT19, GALNT20, ST3GAL1 (e.g. Gene ID: 6482), ST3GAL2 (e.g. Gene ID: 6843), ST3GAL4 (e.g. Gene ID: 6484), ST6GALNAC1 (e.g. Gene ID: 55808), ST6GALNAC3 (e.g. Gene ID: 256435), ST6GALNAC4 (e.g. Gene ID: 27090), or functional variants thereof.

In some embodiments, the vector is a viral vector, a lipid nanoparticle, or a cationic particle.

In some embodiments, the vector is co-administered with a targeting moiety.

### 4. EXAMPLES

### Example 1

Disclosed herein is a demonstration that the brain endothelial glycocalyx is highly dysregulated during aging and neurodegenerative disease. Using bulk and single nucleus RNA sequencing analysis, key pathways of brain endothelial glycocalyx dysregulation in aging and across multiple neurodegenerative disease states (e.g., Alzheimer's disease, Parkinson's disease, frontotemporal dementia, multiple sclerosis, traumatic brain injury, stroke, and amyotrophic lateral sclerosis) are identified. The pathways share a novel “glyco” signature of BBB dysfunction (e.g., abnormal functioning of the glycocalyx, increased BBB permeability, increased brain inflammation, increased age related cognitive decline). Corroborating this BBB dysfunction, significant changes in the bulk structure and composition of the aging mouse brain endothelial glycocalyx were discovered using various microscopy and flow cytometry methods. These results revealed significant perturbation (e.g., abnormal mucin biosynthetic activity in the glycolax layer) in a previously underexplored class of glycoconjugates, known as mucin-domain glycoproteins, a family of densely O-glycosylated proteins with diverse roles in cell surface interactions and cell signaling. Thus, it is shown that aging- and dementia-associated loss of brain endothelial mucins leads to dysregulated transport of blood-derived proteins and small molecules into the brain and, in severe cases, brain bleeding and that restoration of proper BBB function and decrease of neuroinflammation by recovering healthy mucin-type O-glycosylation on the brain endothelium is possible.

In these experiments, the therapeutic approach serves to benefit many age-related, neurological, and vascular disease groups. BBB breakdown is a common hallmark of aging and many neurodegenerative and neurological diseases including Alzheimer's disease, Parkinson's disease, frontotemporal dementia (FTD), multiple sclerosis (MS), traumatic brain injury (TBI), stroke, and amyotrophic lateral sclerosis (ALS). Vascular dysfunction can lead to or exacerbate the decline of brain health in these states by enabling the infiltration of inflammatory and neurotoxic factors into the brain. The approach described herein recovers BBB function and reduces neuroinflammation, and is, thus, an effective therapeutic for a wide range of patients where BBB impairment is implicated.

Methods. The genes of interest were delivered into brain endothelial cells using recombinant AAVs. However, lipid nanoparticles and cationic polymers are also suitable for delivering the genes of interest in DNA or RNA form across cellular membranes into brain endothelial cells.

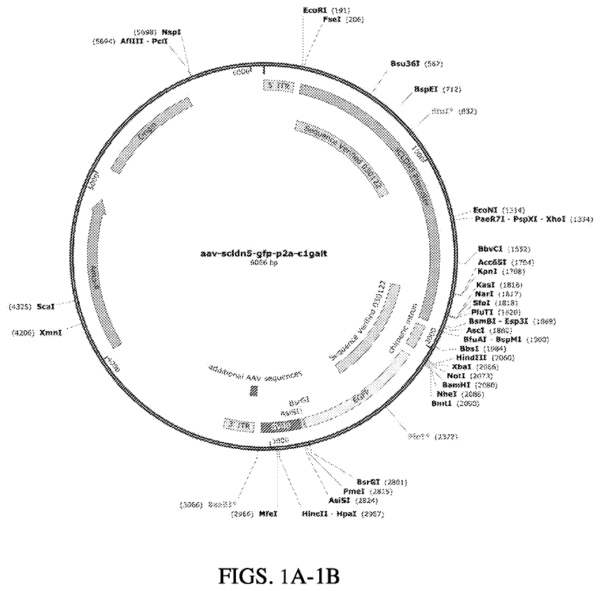

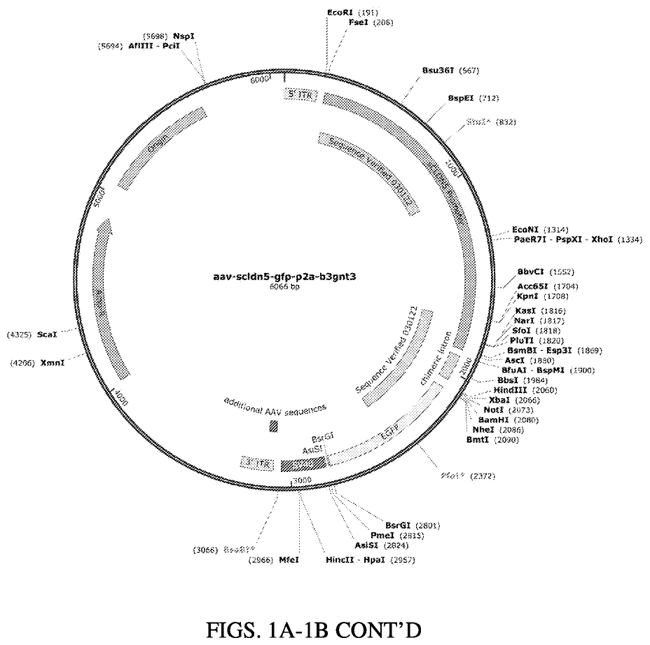

AAV plasmid construction. A recombinant AAV, using AAV-PHP.V1 and an endothelial-specific sCLDN5 promoter, was engineered to develop an AAV that specifically transduced brain endothelial cells. This AAV backbone was used in all virus experiments. FIG. 1A and FIG. 1B provide plasmid maps of the transfer plasmids used to construct the AAVs used to overexpress B3gnt3 and C1galt1 brain endothelium of aged (17-month-old) mice.

In vivo AAV experiments in aged mice and immunohistochemistry. 17-month-old C57BL/6 male mice from the NIA were injected retro-orbitally (RO) with one of three AAVs, AAV-sCLDN5-EFGP, AAV-sCLDN5-EFGP-C1GALT1, or AAV-sCLDN5-EFGP-B3GNT3, at a viral vector dose of with 8×10″ vg per mouse. After 8 weeks, the mice were injected RO with Sulfo-NHS-biotin (Thermo #21335) at 0.25 mg/g bodyweight which was then allowed to circulate for 5 minutes before perfusion with phosphate-buffered saline (PBS). The brains were sectioned at 40 micron thickness and stained with Streptavidin conjugated to Alexa Fluor 647 (Thermo S32357) (1:500) for 2 hr.

Iba1 (Abcam #ab5076) (1:100) and CD68 (BioRad #MCA1957) (1:100) stains were used to evaluate neuroinflammation in the brains of these mice. These are both microglial stains that increase in intensity upon microglial activation, which is an indication of neuroinflammation. Using the mucin-overexpressing AAVs decreased the intensity of both markers. Secondary antibodies used included Donkey anti-Goat-647 (Thermo #A21447) (1:250) and Donkey anti-Rat-555 (Thermo #A48270) (1:250).

All sections were blocked at room temperature for 1.5 hr in 3% normal donkey serum in TBS-T and 0.3% triton-x, stained in primary antibodies overnight (16 hr) in blocking buffer at 4 degrees Celsius, and stained in secondary antibodies for 2 hr in blocking buffer at room temperature before mounting with Prolong Gold Antifade media. All sections were imaged a Zeiss LSM 900 using Z-stacks and quantified using ImageJ.

Results. The results demonstrated that AAV-mediated delivery of two different mucin biosynthetic enzymes, B3GNT3 and C1GALT1, have beneficial effects on BBB integrity and brain health. The work also highlights the following list of brain endothelial mucin genes that are downregulated in mouse aging and human diseases that can be delivered for therapeutic benefit: aging (GALNT1, ST3GAL2, ST6GALNAC4, GALNT18, GALNT2, B4GALT5, ST3GAL1, B3 GNT3, C1GALT1, GALNT10), Alzheimer's Disease (ST3 GAL1, GALNT2, GALNT17, GALNT15, B4GALT5, ST6GALNAC3, GALNT18, C1GALT1), Huntington's Disease (GALNT11, GALNT15, GALNT17, ST6GALNAC3).

### Example 2

Animals. Aged C57BL/6 mice (17-21 months old) were obtained from the National Institute on Aging rodent colony. Young C57BL/6 mice (3 months old) were obtained from Jackson Laboratories. All experiments used male mice. All mice were kept on a 12-h light/dark cycle and provided ad libitum access to food and water. All animal care and procedures complied with the Animal Welfare Act and were in accordance with institutional guidelines and approved by the V.A. Palo Alto Committee on Animal Research and the institutional administrative panel of laboratory animal care at Stanford University.

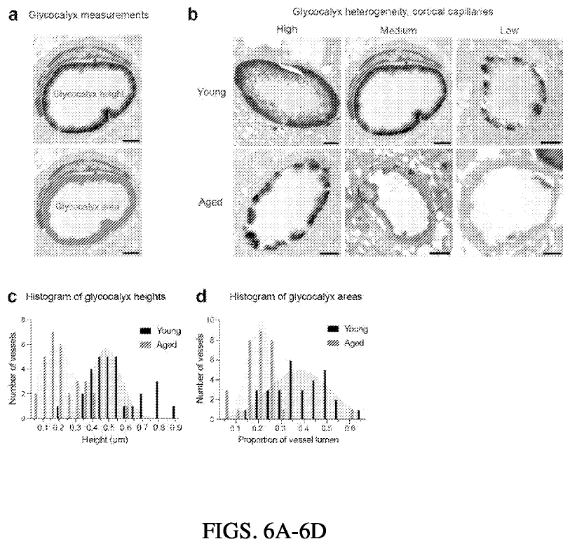

Transmission electron microscopy. Transmission electron micrographs of the brain endothelial glycocalyx layer were obtained as described in a previous study with some modifications⁴. Briefly, young (3-month-old) and aged (21-month-old) mice were perfused with ice-cold fixation buffer composed of 2% glutaraldehyde (EMS), 2% sucrose, 0.1 M sodium cacodylate buffer (EMS), and 2% lanthanum nitrate (EMS) through the left ventricle using a peristaltic pump at 2 mL/min. The brain was removed and sliced coronally using a matrix into 1 mm-thick sections. 1 mm³ cortical punches were cut and immersed in perfusion solution for 2 h before storage overnight in perfusion solution without glutaraldehyde at 0° C. The following day, the samples were washed in a 0.03 M NaOH 2% sucrose solution and subjected to an ascending ethanol gradient (35%, 50%, 70%, 80%, 90%; 10 min each) followed by pure ethanol (2×; 10 min). Samples were progressively infiltrated with Epon resin (EMS), while rocking, and then polymerized at 60° C. for 24-48 h in silicon molds. 90 nm sections were cut using a Leica UC6 ultramicrotome (Leica Microsystems) and collected onto formvar-coated 50-mesh copper grids. The grids were post-stained with 2% uranyl acetate followed by Reynold's lead citrate for 5 min each. Sections were imaged using a Tecnai 12 120 kV TEM (FEI), and data was recorded using either an UltraScan 1000 with Digital Micrograph 3 software (Gatan) or a Rio16 CMOS camera with GWS software (Gatan). Between 6-10 cortical capillaries per animal were captured by a blinded observer. Quantitative analysis of endothelial glycocalyx height and area were performed using ImageJ software (FIG. 6A). For StcE treatment experiments, 3-month-old mice were retro-orbitally injected with 0.25 mg/kg StcE or inactivated StcE^{E447D} 24 h before perfusion.

Flow cytometry. Microvessels from young (3-month-old) and aged (21-month-old) mice were isolated as previously described with some modifications^{26,42}. Briefly, mice were euthanized via CO₂, and brains were retrieved in PBS supplemented with 1% bovine serum albumin (BSA) and 1× cOmplete™ protease inhibitor cocktail (Millipore Sigma) on ice. The olfactory bulb was discarded, and meningeal vessels were removed by gentle rolling on blotting paper. Brains were minced using a razor blade on ice and then homogenized using a loose-fit, 7 mL Dounce (Wheaton) in 1% BSA-PBS with 1× protease inhibitor. The homogenate was centrifuged in 27% (wt/vol) 70 kDa dextran (Sigma) in HBSS at 4400 g for 25 min. Myelin and parenchymal cell layers were removed. Pelleted microvessels were deposited on a pre-wet 40 μm strainer, washed with PBS, and mechanically dissociated into single cells as previously described²⁶. For enzymatic dissociation of brain tissue, previously published protocols were used^{12,13}. Pelleted cells were suspended in FACS buffer (1% BSA in PBS) and stained on ice for 30 min with the following antibodies: rat anti-CD31-PE/CF594 (1:100, BD, 563616), rat anti-CD45-PE/Cy7 (1:200, Biolegend, 103114), mouse anti-heparan sulfate (1:100, Amsbio, clone 10E4, 370255-1), mouse anti-chondroitin sulfate (1:100, Sigma, clone CD-56, C8035), biotinylated hyaluronan binding protein (HABP) (1:150, Amsbio, AMS.HKD-BC41), fluorescein-conjugated Sambucus nigra agglutinin (SNA, 1:300, Vector Labs, FL-1301-2), biotinylated Maackia amurensis agglutinin II (MAA II) (1:300, Vector Labs, B-12651), and StcE^{E447D}-AF647 (5 μg/mL). After washing cells with FACS buffer, secondary incubation with Alexa Fluor-conjugated streptavidin (1:1000, Thermo Fisher Scientific) and secondary antibodies (1:400, Thermo Fisher Scientific) was carried out on ice for 20 min. Live cells were identified using Sytox Blue viability dye (1:1000, Thermo Fisher Scientific, S34857). Flow cytometry analysis was performed on a BD LSRFortessa, and data were analyzed using FlowJo software (TreeStar).

Microvessel imaging. Cerebral microvessels were isolated for IF imaging using the same protocol described above for flow cytometry, except, instead of dissociating microvessels, they were fixed on 40 μm strainers with 4% paraformaldehyde (PFA) in PBS at room temperature for 15 min with gentle rocking. Microvessels were then washed with PBS and mounted on poly-D-lysine-coated slides (Thermo Fisher Scientific). Microvessels were blocked in 3% normal donkey serum (Jackson ImmunoResearch) with 0.3% Triton X-100 (Sigma) in TBS-T (1×TBS with 0.05% Tween20) for 1 h, followed by 1 h incubation at room temperature with primary antibodies. For glyco-profiling of cerebral microvessels, the same primary antibodies/lectins/binding proteins and concentrations from the flow panel were used along with fluorescein-conjugated Vicia villosa agglutinin (VVA, 1:300, Vector Labs, FL-1231-2). For mucin-type O-glycan biosynthetic enzyme staining, rabbit anti-C1galt1 (1:100, Thermo Fisher Scientific, PA5-52814) and rabbit anti-B3gnt3 (1:100, Thermo Fisher Scientific, PA5-21988) were used. For endothelial cell labeling, goat anti-CD31 (1:100, R&D, AF3628) and fluorescein-conjugated Lycopersicon esculentum (Tomato) lectin (LEL, 1:250, Vector Labs, FL-1171-1) were used. For secondary staining, microvessels were washed three times with TBS-T for 5 min each, followed by incubation with Alexa Fluor-conjugated antibodies (1:250, Thermo Fisher Scientific) or streptavidin (1:1000, Thermo Fisher Scientific) for 1 h at room temperature. Microvessels were then washed three times again and coverslipped with Vectashield Hardset Antifade Mounting Medium with DAPI (Vector Labs, H1500-10) or ProLong Gold Antifade Mountant (Thermo Fisher Scientific, P36934). Imaging was performed on a confocal laser-scanning microscope (Zeiss LSM880). All observed single-plane microvessels were captured and then quantified using ImageJ software.

Luminal vascular StcE^{E447D}-AF647 and SNA-Cy3 staining. For luminal vascular labeling, mice were euthanized with 2.5% (v/v) Avertin and transcardially perfused via peristaltic pump at 2 mL/min with the following ice-cold solutions: 8 mL PBS, 10 mL of 5 μg/mL of StcE^{E447D}-AF647 or SNA-Cy3 (Vector Labs, CL-1303-1), and 8 mL of 4% PFA. Brain, heart, and liver were extracted and post-fixed in 4% PFA at 4° C. overnight before preservation in 30% sucrose in PBS. Tissues were sectioned into 40 μm slices using a microtome. Slices were subsequently blocked in 3% normal donkey serum with 0.3% Triton X-100 in TBS-T for 1.5 h at room temperature and incubated with goat anti-CD31 (1:100, R&D, AF3628) at 4° C. overnight. The following day, slices were washed three times with TBS-T, stained with Alexa Fluor-conjugated donkey anti-goat secondary antibody (1:250, Thermo Fisher Scientific) for 1.5 h at room temperature, washed three times again, mounted, and coverslipped with Vectashield Hardset Antifade Mounting Medium with DAPI (Vector Labs, H-1500-10) before imaging on a confocal laser-scanning microscope (Zeiss LSM880). Images were analyzed using ImageJ software, and luminal vascular coverage was calculated as vessel (CD31′) area occupied by StcE^{E447D}-AF647 or SNA-Cy3 divided by total vessel area.

RNA-seq glyco-gene analysis. Aging and neurodegenerative disease RNA-seq datasets demonstrating robust brain endothelial cell enrichment were chosen for glyco-gene analysis. Glyco-genes based on KEGG listed glycosylation enzymes and related proteins were filtered. Most glycoproteins were excluded due to the enormous variety of members in this family which possess biological functions not directly relevant to glycosylation. Significantly upregulated and downregulated glyco-genes in each dataset were used for Reactome pathway analysis with p_{adj}<0.05 set as the threshold for significant enrichment.

Luminal cerebrovascular proteome enrichment and peptide preparation. Young (3-month-old) and aged (21-month-old) mice (n=6 mice/group) were euthanized with 2.5% (v/v) Avertin and transcardially perfused via peristaltic pump with the following ice-cold solutions: 8 mL of PBS, 20 mL of 0.5 mg/mL Sulfo-NHS-biotin (Thermo Fisher Scientific, 21217), and 10 mL of 50 mM Tris-PBS. Cerebral microvessels were isolated as described in earlier sections and collected for lysis via sonication in RIPA buffer (Thermo Fisher Scientific) supplemented with 1× protease inhibitor. Lysates were centrifuged at 13,000 g for 15 min at 4° C., and supernatant was saved for downstream enrichment. Protein concentration was measured by BCA (Pierce), and 1 mg of each sample was incubated with 70 μL of streptavidin magnetic beads (Thermo Fisher Scientific, 88817) at 4° C. overnight. The following day, beads were processed for proteomic analysis using a published protocol with some modificiations⁴³. Briefly, beads were washed 1× with 200 μL of 50 mM Tris-HCl (pH 7.5) and 2× with 200 μL of 2 M urea in 50 mM Tris (pH 7.5) and incubated in 80 μL 2 M urea in 50 mM Tris with 1 mM dithiothreitol (DTT, Thermo Fisher Scientific, R0861) and 0.4 ug trypsin/LysC (Fisher, V5073) at 25° C. for 1 h shaking at 1000 rpm. Supernatant was further reduced with 4 mM DTT for 30 min with shaking and alkylated with 10 mM iodoacetamide (IAA, Sigma, 11149) for 45 min in the dark with shaking. An additional 0.5 ug of trypsin/LysC was added to samples for overnight digestion at 37° C. with shaking. The following day, samples were acidified to 1% vol/vol formic acid and desalted using BioPureSPN mini C18 columns (Nest Group). All centrifugation steps were carried out at 50 g for 1 min at room temperature. Columns were washed with 200 μL HPLC-grade methanol (Fisher) and equilibrated two times with 200 μL 0.1% formic acid in HPLC-grade water (Fisher) (“solvent A”). Samples were loaded onto columns, washed four times with solvent A, and eluted two times with 75 μL 0.1% formic acid in 80% acetonitrile (“solvent B”). The combined elutes were dried in a vacuum concentrator and reconstituted in 10 μL solvent A for LC-MS/MS.

Mass spectrometry proteomics. LC-MS/MS analysis was performed on a Q Exactive HF-X (Thermo Fisher Scientific) with an UltiMate 3000 RSLCnano system (Thermo Fisher Scientific). Peptides were loaded on an in-house 75 μm (inner diameter) capillary column packed with 40 cm of ReproSil-Pur 120 C18-AQ 1.9 Nm resin (Dr. Maisch). Chromatographic separation was achieved using a flow rate of 300 nL/min with the following 120 min gradient: 96% A+4% B for 18 min, 70% A+30% B for 72 min, 60% A+40% B for 15 min, and 4% A+96% B for 15 min, where solvent A was 0.1% formic acid in HPLC-grade water (Fisher) and solvent B was 0.1% formic acid in HPLC-grade acetonitrile (Fisher). Full MS scans were acquired at a resolution of 60,000, with an automatic gain control (AGC) target of 3e6, maximum injection time (IT) of 20 ms, and scan range 300-1650 m/z in a data-dependent mode. MS2 scans were acquired with the following parameters: resolution of 15,000, AGC target of 1e5, maximum IT of 54 ms, loop count 15, TopN 15, isolation window 1.4 m/z, fixed first mass 100.0 m/z, normalized collision energy (NCE) 28 units, charge exclusion of unassigned, 1, 6-8, and >8, peptide match preferred, exclude isotopes on, and fragmented m/z values were dynamically excluded from further selection for a period of 45 s. Raw data were processed and analyzed using MaxQuant and Perseus⁴⁴. In brief, peptide spectral matches were made against a target-decoy Mus musculus reference proteome database downloaded from Uniprot. Methionine oxidation and N-terminal acetylation were specified as variable modifications, and carbamidomethylation of cysteines was specified as a fixed modification. Precursor ion search tolerance of 20 ppm and product ion mass tolerance of 20 ppm were used for searches. Both unique and razor peptides were used for quantification. Results were filtered to a 1% FDR at the peptide and protein levels. Proteins were quantified and normalized using MaxLFQ⁴⁵ with a label-free quantification (LFQ) minimum ratio count set to 1. For quantitative comparative analysis, protein intensity values were log 2-transformed, and missing values were imputed from a normal distribution with width 0.3 and downshift value of 1.8 using Perseus. Principal component analysis (PCA) analysis was performed in Perseus using the Benjamini-Hochberg FDR with a cutoff of 0.05. GO term enrichments were performed using DAVID⁴⁶ with the Mus musculus proteome as a background.

In vivo StcE treatment and cerebral bleeding assessment. All recombinant StcE proteins were produced as described in previous publications^{15,24}. For in vivo experiments, proteins were run through Pierce high-capacity endotoxin removal columns (Thermo Fisher Scientific) at least seven times following manufacturer's instructions. Endotoxin levels were tested using HEK-Blue™ LPS Detection Kit 2 (InvivoGen) according to manufacturer recommendations. Mice were injected retro-orbitally with 0.25 mg/kg StcE once a day for 2 days before perfusion with ice-cold PBS. Cerebral bleeding was visualized by eye post-perfusion and by hematoxylin & eosin (H&E) staining. For H&E staining, hemibrains were formalin-fixed and paraffin embedded (FFPE) and cut into 5 μm thick sagittal sections mounted on slides. Sections were deparaffinized in xylene (3×; 3 min), hydrated in a series of graded alcohols (2×100%, 1×95%, 1×80%; 3 min each), and stained with Richard Allan hematoxylin (4 min; followed by 30 s in 4% acetic acid in water and dipping in 0.3% ammonia water) and eosin with phloxine (30 s). Sections were then dehydrated (10 dips in 95% ethanol followed by 2×1 min in 100% ethanol), cleared in xylene (3×; 1 min), and coverslipped prior to imaging on a widefield microscope (Zeiss Axiolmager).

BBB leakage assay. Mice were anesthetized and injected retro-orbitally with Sulfo-NHS-biotin (Thermo Fisher Scientific, 21335) at 0.25 mg/g body weight. The tracer was allowed to circulate for 5 min before perfusion with PBS. Hemibrains were post-fixed in 4% PFA overnight at 4° C., cryopreserved in 30% sucrose, and sagittally sectioned into 40 μm slices. Sections were blocked and co-stained with CD31 and the appropriate secondary antibody as described earlier and Alexa Fluor 647-conjugated streptavidin (1:1000, Thermo Fisher Scientific). Images were taken on a confocal laser-scanning microscope (Zeiss LSM880) and analyzed using ImageJ software. Multi-colored gradient images were generated using the fire LUT in ImageJ. Permeability index of vessels was determined as the area occupied by tracer divided by the vessel area as previously described.

Immunohistochemistry. Mice were euthanized with 2.5% (v/v) Avertin and perfused with PBS. Hemibrains were fixed in 4% PFA at 4° C. overnight before preservation in 30% sucrose in PBS. Brains were sectioned into 40 μm slices which were subsequently blocked in 3% normal donkey serum with 0.3% Triton X100 in TBS-T for 1.5 h at room temperature and incubated at 4° C. overnight with the following primary antibodies: goat anti-CD31 (1:100, R&D, AF3628), goat anti-Iba1 (1:100, Abcam, ab5076), rat anti-CD68 (1:100, Bio-Rad, MCA1957), goat anti-collagen type IV (1:100, Sigma, AB769), and rat anti-NID1 (1:100, Thermo Fisher Scientific, MA1-06501). Slices were washed, stained with the appropriate Alexa Fluor-conjugated secondary antibodies (1:250, Thermo Fisher Scientific) for 2 h at room temperature, mounted, and coverslipped with Vectashield Hardset Antifade Mounting Medium with DAPI. Imaging was performed on a confocal laser-scanning microscope (Zeiss LSM880), and images were analyzed using ImageJ.

Design and cloning of pAAV-sCLDN5 constructs. The Ple261 MiniPromoter (“sCLDN5”) pEMS1938 was a gift from Elizabeth Simpson (Addgene plasmid #82563). A cis rAAV genome plasmid with AAV2 inverted terminal repeats was utilized for cloning of a sCLDN5 and EGFP reporter using restriction enzymes and In-Fusion Snap Assembly (Takara Bio). To knockdown C1galt1 in brain endothelial cells, de novo predictions of siRNA guides targeting C1galt1 were generated using the DSIR algorithm⁴⁹ and subsequently filtered using “Sensor rules” to select for sequences with highly favorable shRNA features. Three de novo 97-mer miR-E shRNA sequences (Table 1) were synthesized (IDT) and inserted into pAAV-sCLDN5-EGFP using restriction enzyme cloning for in vitro evaluation. To overexpress C1GALT1 and B3GNT3 in brain endothelial cells, P2A-C1GALT1 and P2A-B3GNT3 were cloned into pAAV-sCLDN5-EGFP using restriction enzyme cloning to generate pAAV-sCLDN5-EGFP-P2A-C1GALT1 and pAAV-sCLDN5-EGFP-P2A-B3GNT3, respectively.

In vitro pAAV evaluation. The mouse brain endothelial cell line bEnd.3 (ATCC, CRL-2299) was cultured in high glucose DMEM (Thermo Fisher Scientific, 10567022) supplemented with 10% fetal bovine serum (FBS) and 1% penicillin/streptomycin and maintained in a humidified incubator containing 5% CO₂ at 37° C. bEnd.3 cells were split into a 24-well plate 1 day before transfection. Cells at approximately 70% confluency were transfected with the pAAVs described in the previous section via lipofectamine 3000 according to the manufacturer's recommendations (Thermo Fisher Scientific, L3000008). After 16 h, cell media was replaced with fresh, pre-warmed media. 2.5 days after transfection, cells were collected from plates using enzyme-free cell dissociation buffer (Thermo Fisher Scientific, 1315014) and resuspended in FACS buffer. Cells were stained with StcE^{E447D}-AF647 (5 μg/mL) on ice for 20 min. Live cells were identified using Sytox Blue viability dye (1:1000, Thermo Fisher Scientific, S34857). Flow cytometry analysis was performed on a Sony SH800S sorter, and data were analyzed using FlowJo software (TreeStar). Only live EGFP⁺ cells were used for analysis of StcE^{E447D}-AF647 MFI.

AAV production. For production of PHP.V1-sCLDN5::EGFP and all other plasmids containing the PHP.V1 (pUCmini-iCAP-PHP.V1 was a gift from Viviana Gradinaru, Addgene plasmid #127847) capsid, AAV production was performed in-house utilizing a previously published protocol⁵². In brief, triple transfection of HEK293T cells was performed on 90-95% confluent cells in DMEM containing glutamax supplemented with 5% FBS and non-essential amino acids. Fresh, warm media was replaced 12 h post-transfection. Media was collected 72 h post-transfection. Fresh, warm media was added and collected along with cells 120 h post-transfection and combined with the previous fraction. Cells and media were centrifuged at 2,000 g for 15 min at room temperature. Supernatant was collected in a separate bottle and combined with 40% (wt/vol) PEG (final concentration 8% wt/vol PEG) and incubated on ice for 2 h before transferring to 4° C. overnight. Cell pellet was re-suspended in buffer containing salt-active nuclease (SAN, ArcticZymes Technologies) and incubated at 37° C. for 1 h before transferring to 4° C. overnight. PEG-media was centrifuged at 4,000 g for 30 min at 4° C. After centrifugation, supernatant was bleached and discarded. PEG pellet was resuspended in SAN+SAN buffer, combined with the previous fraction, and incubated at 37° C. for an additional 30 min. Lysate was centrifuged at 2,000 g for 15 min at room temperature, and supernatant was loaded onto an iodixanol gradient (15%, 25%, 40%, and 60% fractions). Gradients were transferred to an ultracentrifuge (Beckman Coulter) using a Type 70 Ti rotor set at 350,000 g for 2 h and 25 min at 18° C. AAV particles were collected from the 40/60% interface, washed in PBS, and concentrated using an Amicon Ultra-15 (Millipore Sigma) filter device with a 100 kDa cutoff. AAV titration was performed using the AAVpro Titration Kit (for Real Time PCR) Ver.2 (Takara Bio). AAVs were injected retro-orbitally at 8×10¹¹ vg per mouse.

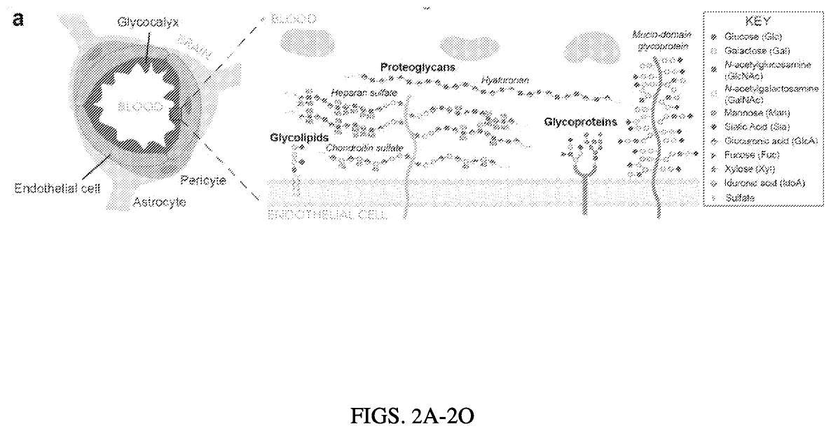

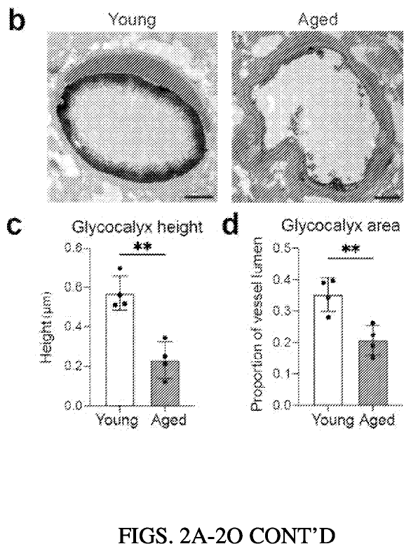

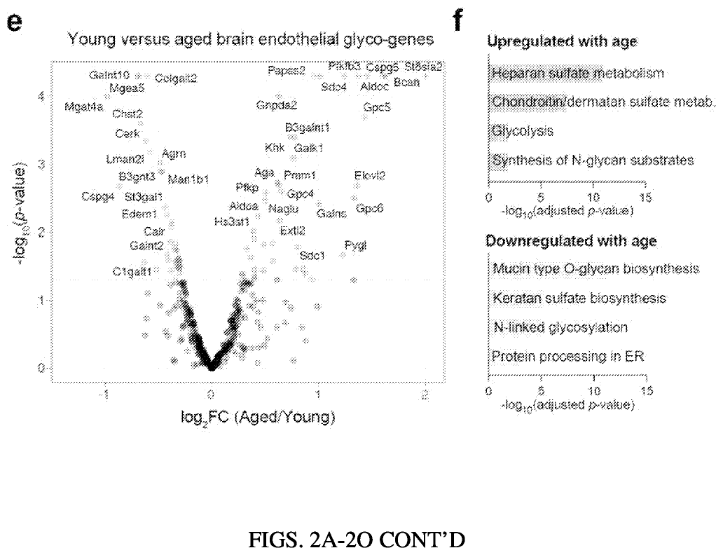

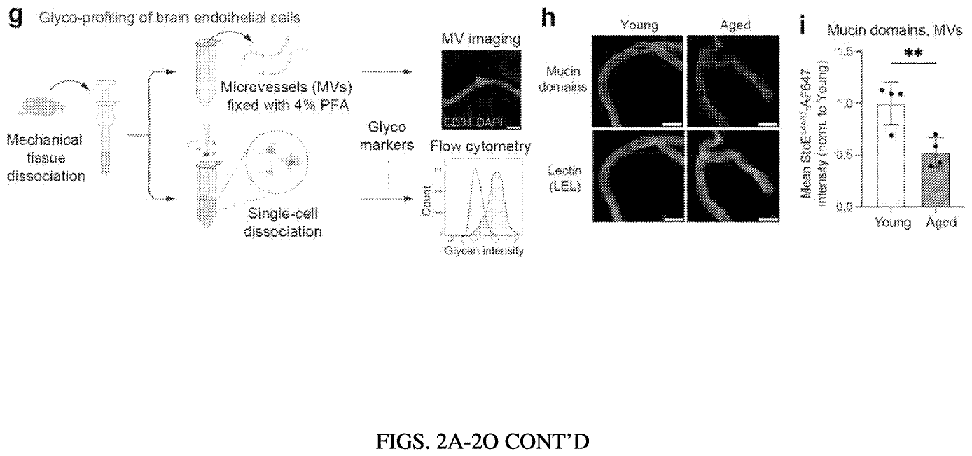

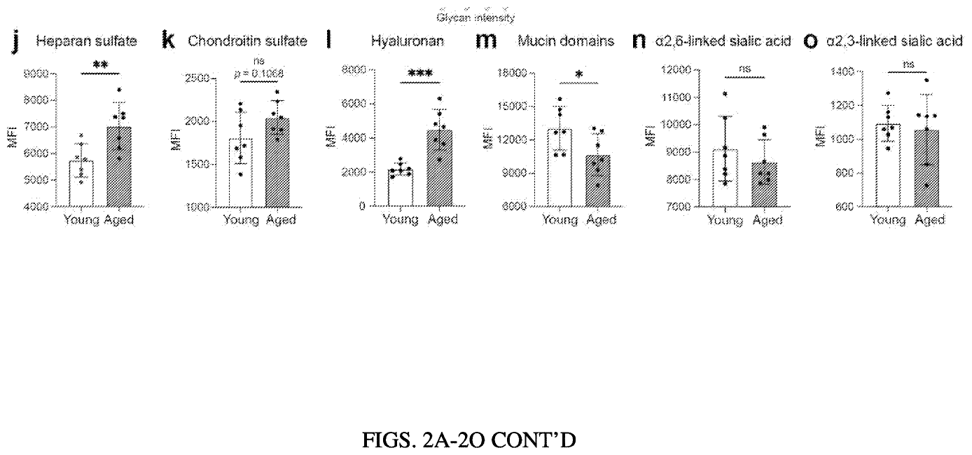

Statistical analysis. Data handling and statistical analyses were performed using R studio and GraphPad Prism Software unless stated otherwise. All statistical analyses comparing measurements between two groups were carried out using unpaired two-tailed Student's t-tests. Ap-value of <0.05 was considered significant. All experimental procedures involving multiple experimental groups were carried out in an alternating fashion when possible to avoid temporal and technical biases. Data in FIGS. 2B-2D, 2J-20, 3B-3E, 4E-4M, 5G-5L 7A, 8A-8D, and 12A were successfully replicated in at least two independent experiments.

Data availability. Raw mass spectrometry data generated in this study have been deposited in the PRIDE database.

The blood-brain barrier (BBB) forms a tightly regulated vascular interface between the blood and brain that is essential for supporting proper brain function. The highly restrictive nature of the BBB is enabled by the unique properties of brain endothelial cells, including specialized tight junctions, exceptionally low rates of transcytosis, and selective influx and efflux transporters. A commonly overlooked, yet integral component of the BBB that is increasingly being recognized as an additional key contributor to BBB function is the brain endothelial glycocalyx layer. The glycocalyx layer is a complex meshwork of glycans and glycoconjugate species (e.g., proteoglycans, glycoproteins, and glycolipids) that coats the luminal surface of the BBB and, more broadly, mediates numerous cell surface processes including cell signaling, adhesion, transport, and morphology (FIG. 2A). Despite forming the BBB's first barrier-to-entry for any blood-derived factors, very little is known about the composition and functional roles of the brain endothelial glycocalyx layer. Furthermore, BBB dysfunction, characterized by increased vascular “leakiness” to neurotoxic and inflammatory circulating factors, is a key pathological hallmark in aging and eurodegenerative diseases. However, studies investigating the significance of the glycocalyx in this process are lacking. The structural and compositional aging- and disease-associated changes in the brain endothelial glycocalyx was profiled to determine how glycocalyx dysregulation in these states may contribute to BBB dysfunction.

Cerebrovascular glycocalyx dysregulation during aging. Transmission electron microscopy (TEM) with lanthanum nitrate staining to visualize brain endothelial glycocalyx layers in young (3-month-old) and aged (21-month-old) mice was used to characterize aging-associated bulk structural changes in the brain endothelial glycocalyx layer. While traditional electron microscopy of the BBB does not enable visualization of the glycocalyx layer, incorporation of cationic metal stains, like lanthanum nitrate or ruthenium red, into samples reveals a substantial layer of glycans on the vascular lumen (FIG. 2B). A significant reduction in luminal glycocalyx layers was observed in cortical capillaries of aged mice compared to young (FIGS. 2B-2D; FIGS. 6B-6D). Quantitative analyses showed decreases in both average glycocalyx thickness (0.540±0.086 μm vs. 0.232±0.092 μm) and average glycocalyx area (0.367±0.054 and 0.207±0.047 as a proportion of lumen area) with aging (FIGS. 2B-2D; FIGS. 6A-6D).

A bulk RNA-sequencing (RNA-seq) dataset of brain endothelial cells collected from young (3-month-old) and aged (19-month-old) mice to identify classes of glycoconjugates (FIG. 2A) that might contribute to these aging-associated aberrations in the glycocalyx. Significant dysregulation of numerous glycosylation-related genes (glyco-genes) with aging (FIG. 2E) was identified. Pathway analysis revealed notable upregulation of genes involved in heparan sulfate metabolism (Sdc4, Hs3st1, Extl2, Gpc5) and downregulation of genes involved in mucin-type O-glycan biosynthesis (Galnt10, B3gnt3, Galnt2, C1galt1) in the aged brain endothelium compared to young (FIGS. 2E-2F).

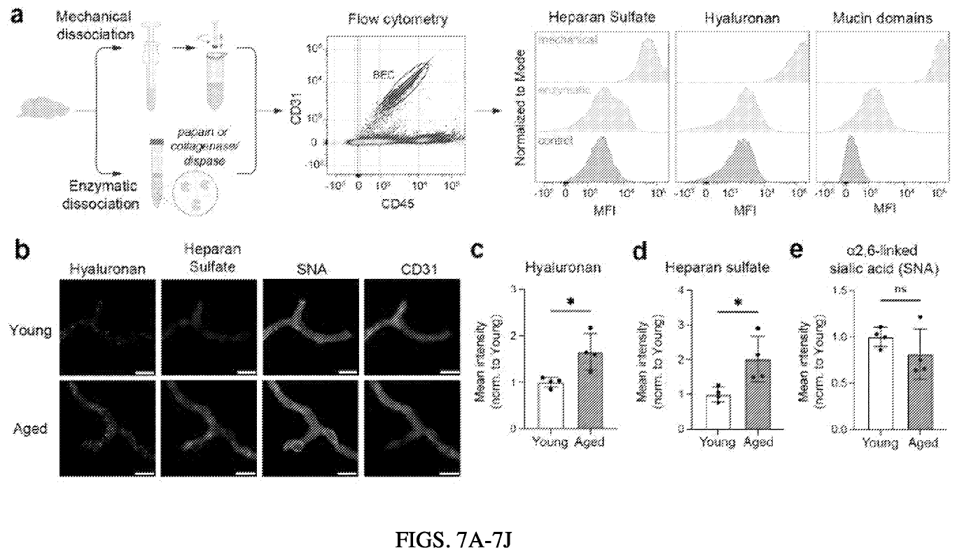

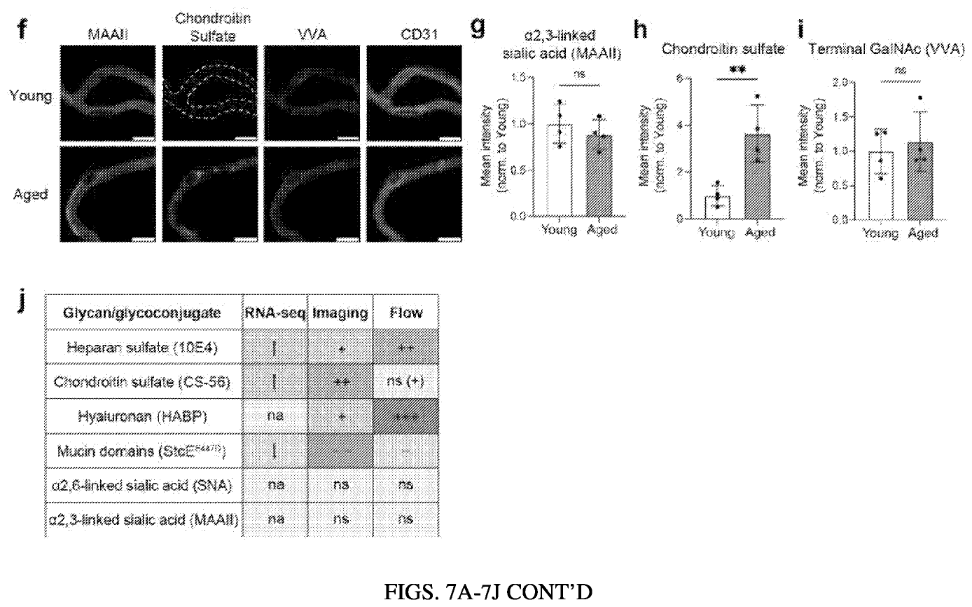

The glycocalyx via imaging and flow cytometry-based assays, using a panel of antibodies and binding proteins selective for several major classes of glycans, glycoconjugates, and specific monosaccharides, were profiled to determine if these transcriptional changes were reflected at the level of glycocalyx composition (FIG. 2G). The brain's extensive extracellular matrix precluded reliable endothelial-specific quantification of many glycan and glycoconjugate species in intact brain slices using traditional immersion staining methods. Methods to dissociate brain tissue into microvessels or single cells were optimized and it was consistently found that mechanical dissociation methods better preserved brain endothelial glycocalyx staining compared to commonly used enzymatic dissociation methods (FIG. 2G; FIG. 7A). Fluorescence imaging of acutely isolated microvessels from young and aged mice revealed significantly increased expression of hyaluronan (via hyaluronan binding protein (HABP)), heparan sulfate (via 10E4 antibody), and chondroitin sulfate (via CS-56 antibody), decreased expression of mucin-domain glycoproteins (via StcE^{E447D}), and no significant change in α2,6- or α2,3-linked sialic acids (via SNA and MAAII, respectively) or terminal N-acetylgalactosamine (via VVA) with aging (FIG. 2H-2I; FIGS. 7B-7I). Flow cytometry analysis of acutely isolated brain endothelial cells demonstrated congruent results, which were also largely consistent with the dysregulated glycosylation pathways identified in the RNA-seq analysis (FIGS. 2J-20; FIG. 7J). Together, these data demonstrated that the brain endothelial glycocalyx undergoes substantial structural and compositional alterations during aging, which may in part be dictated by transcriptional changes. Hyaluronan and heparan sulfate may have detrimental effects on BBB function and Alzheimer's disease (AD) pathology, implicating potentially negative consequences of their upregulation with age. However, downregulation of mucin-type O-glycosylation is not linked to aging-associated BBB impairment.

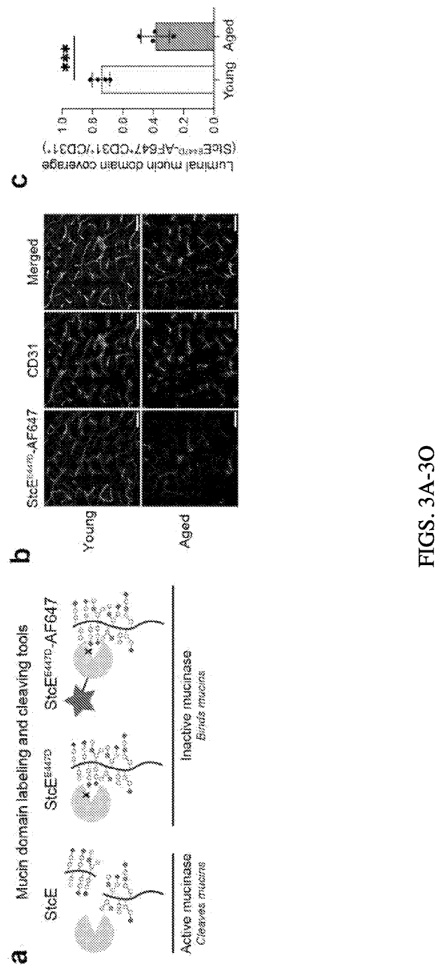

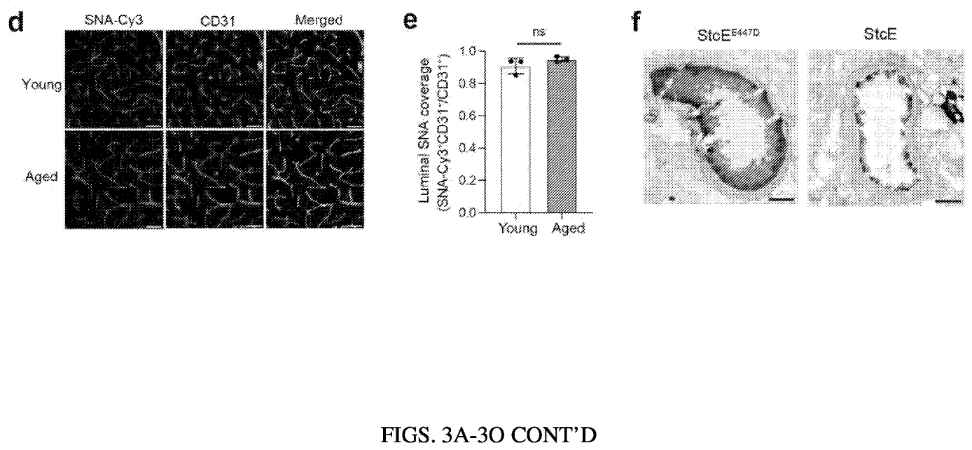

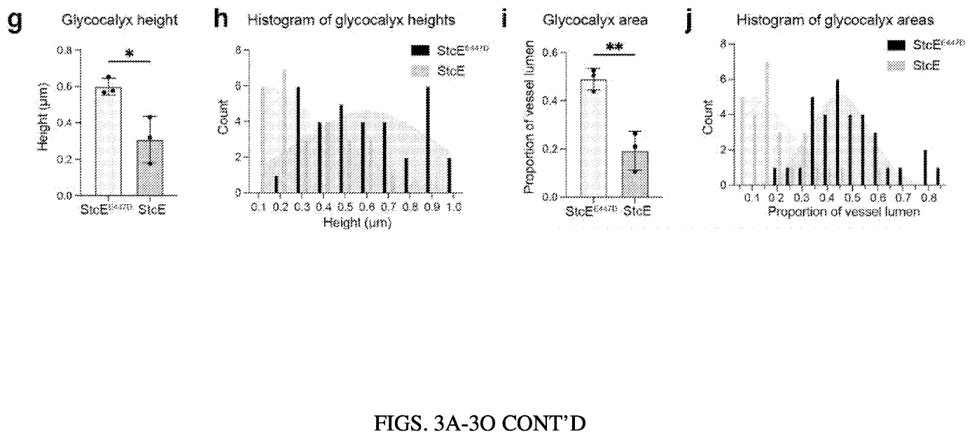

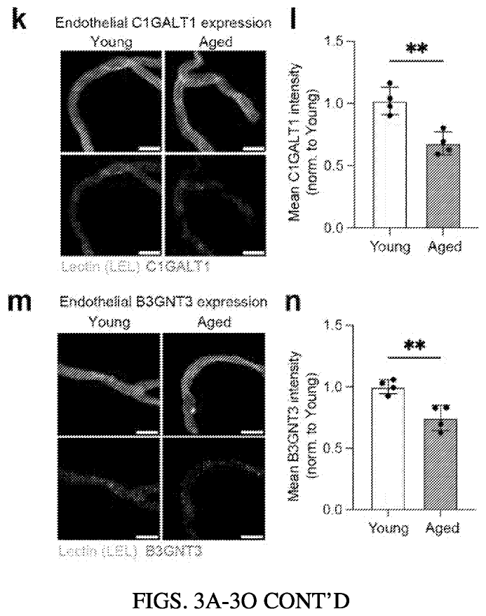

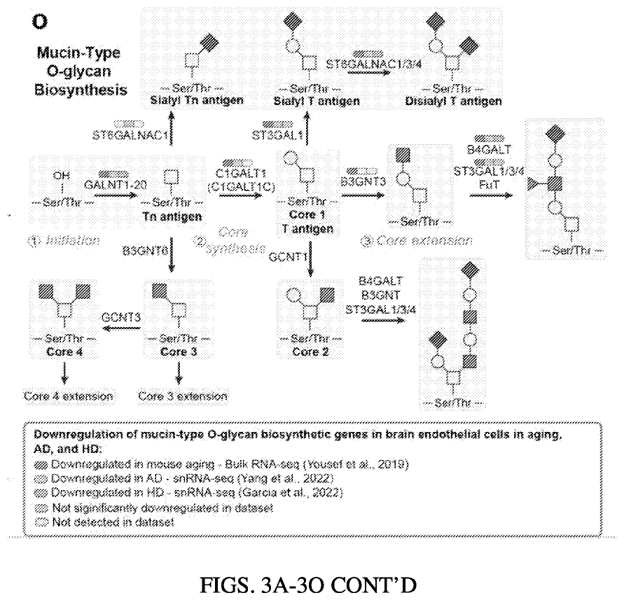

Downregulated mucin-type O-glycosylation in aging and disease. Mucin-type 0-glycosylation is a post-translational modification consisting of glycans initiated by an α-N-acetylgalactosamine (α-GalNAc) attached to proteins via serine and threonine residues (FIG. 30). Mucin-domain glycoproteins contain modular regions of dense mucin-type 0-glycosylation which adopt unique extended bottlebrush structures and mediate many diverse cell surface processes (FIG. 2A). A set of recombinant mucin-selective proteins derived from a broadly cleaving mucinase from E. coli known as secreted protease of C1 esterase inhibitor (StcE) was used to evaluate the contribution of mucin-domain glycoproteins to the brain endothelial glycocalyx layer (FIG. 3A). StcE has a distinct peptide- and glycan-based cleavage motif that is limited to mucin domains. Taking advantage of this, Alexa Fluor 647 (AF647) conjugated catalytically inactivated StcE (StcE^{E447D}-AF647) was developed and used to label luminal mucin-domain glycoproteins, unconfounded by abluminal glycoproteins, via intracardial perfusion. The technique revealed strong luminal mucin-domain glycoprotein staining in young mice and weaker, more heterogeneous staining in aged mice (FIGS. 3B-3C).

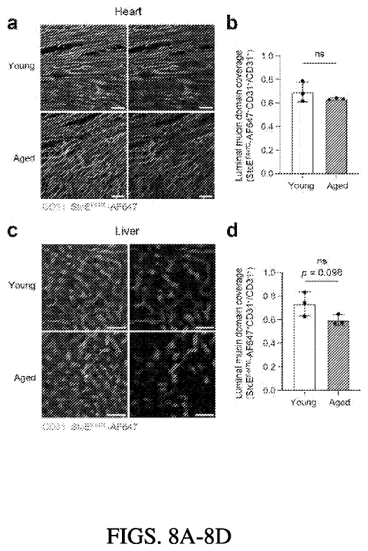

The age-dependent loss of luminal StcE^{E447D}-AF647 signal appeared to be particular to the brain; little to no differences in luminal mucin domain coverage in the heart or liver with age were observed (FIG. 8A-8D).

Despite some overlapping substrate specificity between StcEE447D and Sambucus nigra lectin (SNA), perfusion of Cy3 conjugated SNA (SNA-Cy3) showed no significant differences in luminal cerebrovascular labeling between young and aged mice (FIGS. 3D-3E). Furthermore, intravenous injection of 0.25 mg/kg of enzymatically active StcE in young mice induced significant loss of the brain endothelial glycocalyx layer in 24 h as visualized using TEM with lanthanum nitrate staining (FIGS. 3F-3J).

The data suggested that mucin-domain glycoproteins are crucial structural constituents of the brain endothelial glycocalyx layer and that their decline with aging contributes to the reduction of the glycocalyx layer observed in FIG. 2B. The age-dependent loss of mucin domain labeling via StcE^{E447D}-AF647 could be due to either a decrease in mucin-type 0-glycosylation, as suggested by the bulk RNA-seq data (FIGS. 2E-2F), or a decrease in the mucin-domain glycoprotein scaffolds themselves.

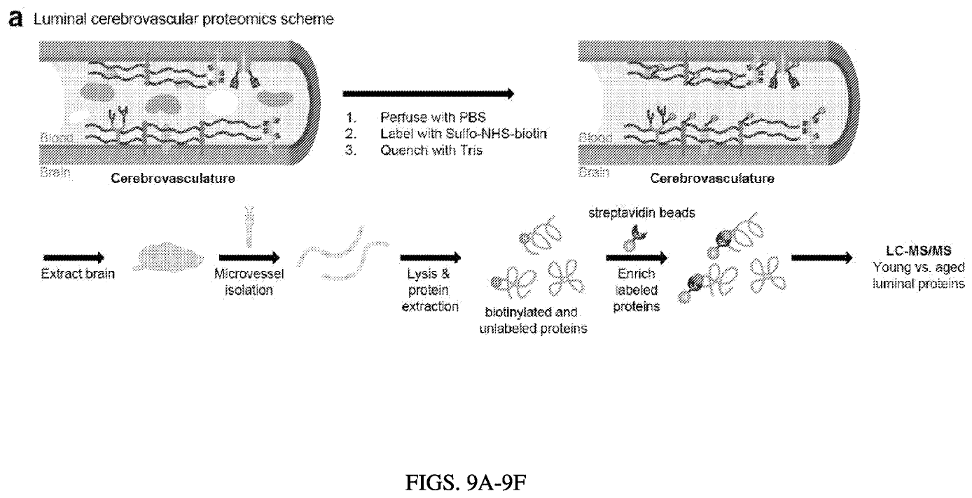

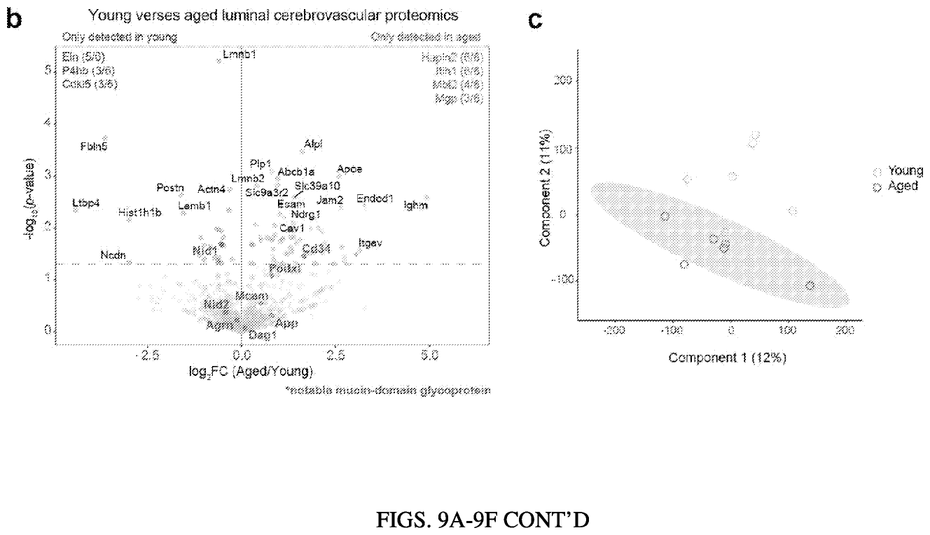

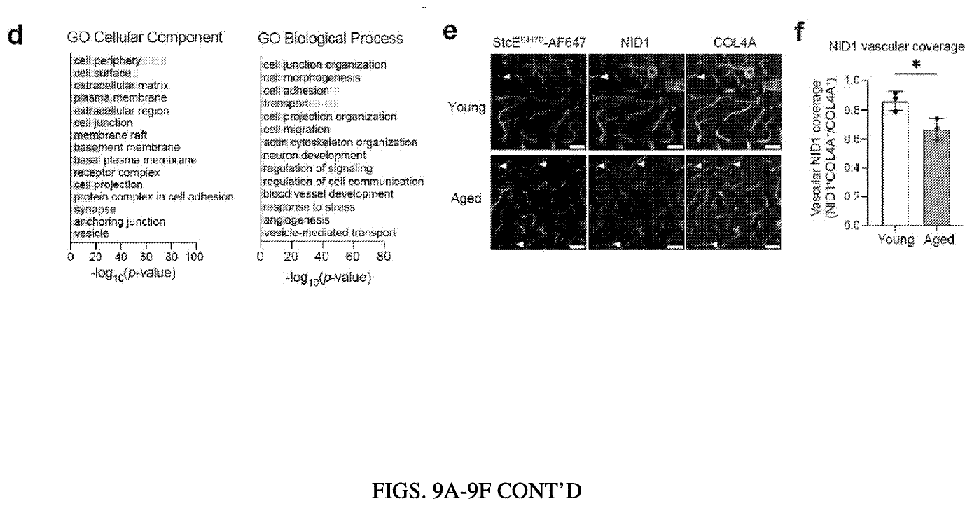

To test the latter, the luminal cerebrovascular proteome of young and aged mice were compared by perfusing mice with sulfo-NHS-Biotin to chemically modify luminal proteins with a biotin tag which were subsequently enriched for mass spectrometry-based proteomic analysis (FIG. 9A).

One-thousand and eighty (1080) unique proteins were identified and gene ontology (GO) term analysis verified robust enrichment of proteins localized to the cell membrane and periphery and involved in cell surface processes including cell junction organization, adhesion, and transport (FIGS. 9B-9D).

In addition, several age-upregulated brain endothelial cell surface proteins, including ALPL and CAVI, were substantiated by the data. However, NID1 was the only significantly downregulated known mucin-domain glycoprotein detected in the dataset, but the loss of luminal StcE^{E447D}-AF647 signal in aged mice did not correlate with a loss of NID1 signal (FIGS. 9B, 9E-9F). While the availability of mucin-domain glycoprotein annotations and LC-MS/MS detection limits is limited, an obvious loss of mucin-domain glycoprotein scaffolds with age on the vascular lumen were not found.

On the other hand, decreased levels of mucin-type 0-glycan biosynthetic enzymes C1GALT1 and B3GNT3 were observed in acutely isolated cerebral microvessels from aged mice compared to young, suggesting that downregulation of mucin-type O-glycosylation contributes to the loss of mucin-domain glycoprotein staining observed with age (FIGS. 3K-3N).

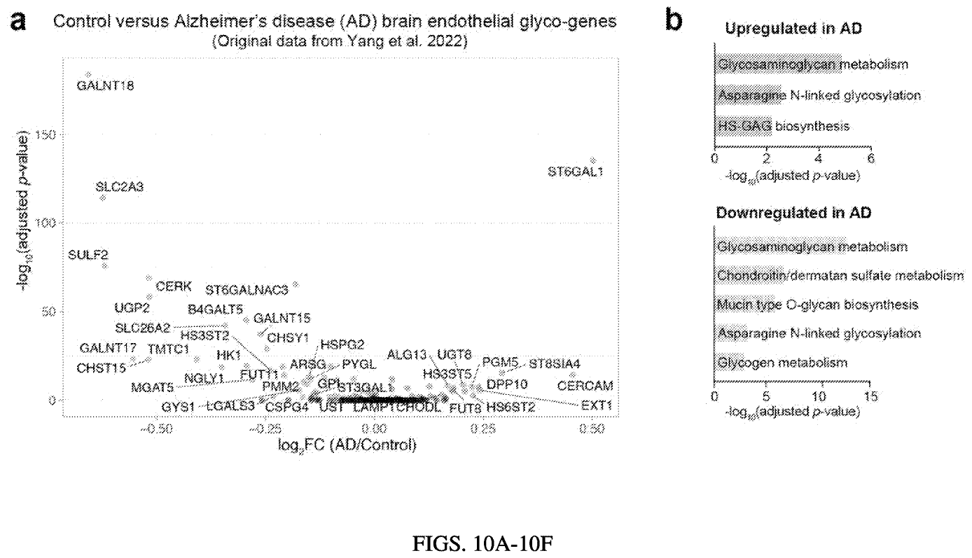

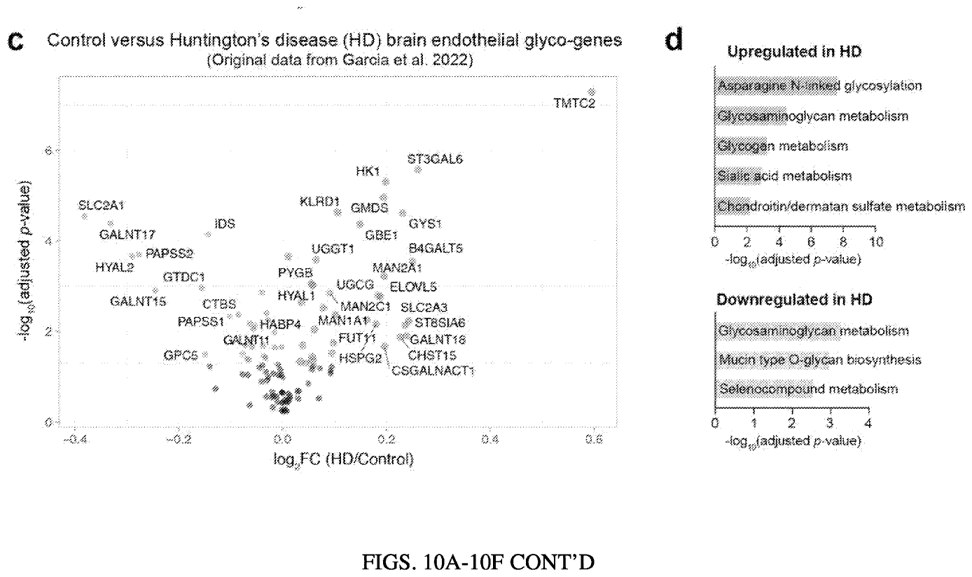

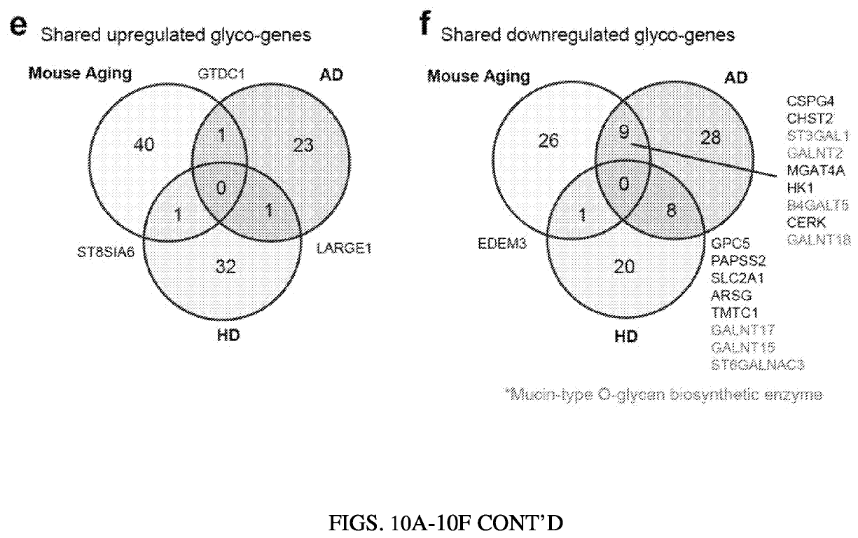

Whether similar pathways of brain endothelial glycocalyx dysregulation seen in the aging dataset could be observed in different neurodegenerative diseases was sought to be determined. Differential glyco-gene analysis was conducted on single-nucleus RNA-seq (snRNA-seq) datasets of Alzheimer's disease (AD) and Huntington's disease (HD), and it was found that brain endothelial mucin-type O-glycan biosynthesis to be a shared downregulated pathway enriched in both disease datasets (FIGS. 10A-10D). While very few upregulated glyco-genes overlapped between any two datasets (aging, AD, or HD), approximately one-third of all significantly downregulated glyco-genes in any dataset was shared by one other dataset (FIGS. 10E-10F). Of the 18 shared downregulated genes, 7 of them were mucin-type 0-glycan biosynthetic enzymes (FIG. 10F; FIG. 30). The data suggested that downregulation of mucin-type O-glycosylation contributes to a conserved glyco-signature of BBB dysfunction seen across multiple neurodegenerative states.

Decreased mucin-type O-glycosylation promotes BBB dysfunction. To examine the effects of downregulated mucin-type O-glycan biosynthesis on BBB function, a brain endothelial cell-specific AAV targeting approach was used to specifically knock down C1galt1

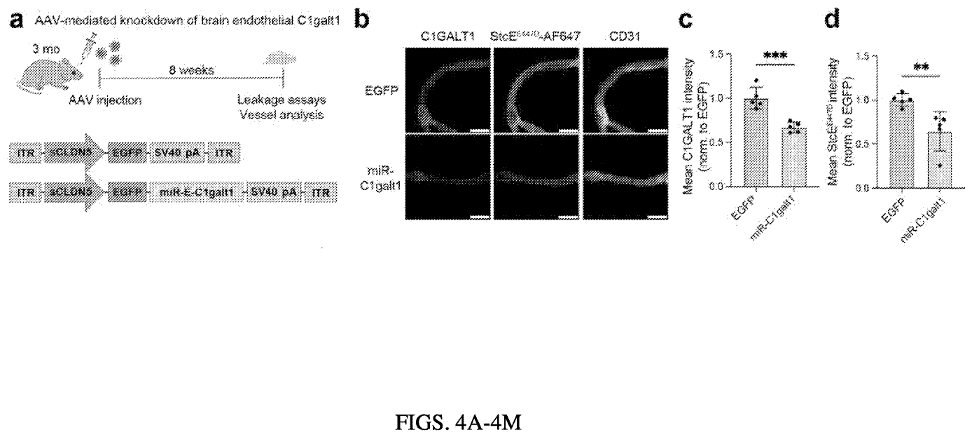

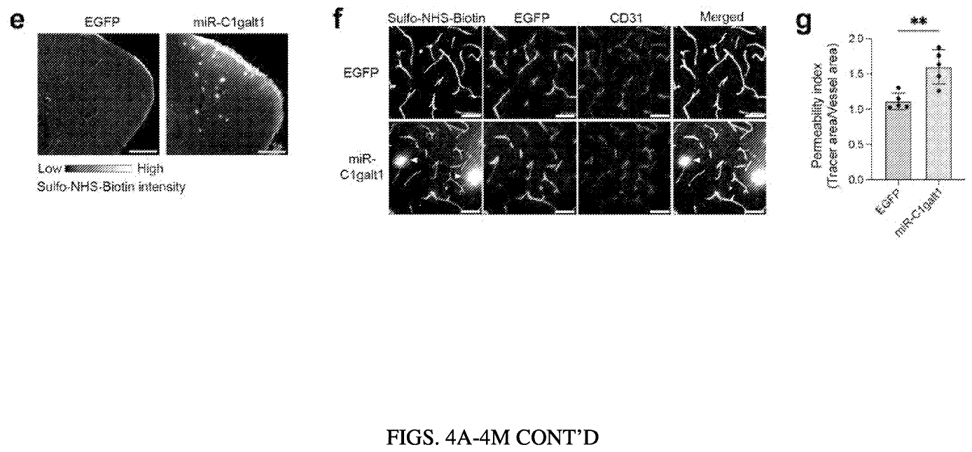

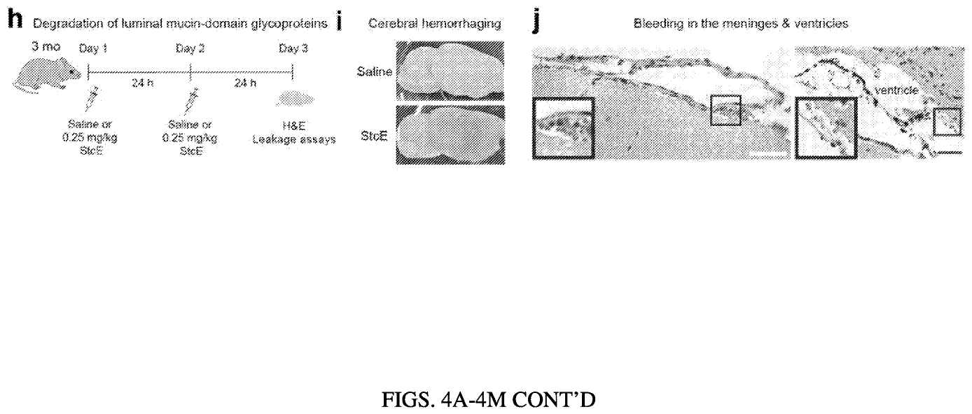

in brain endothelial cells in vivo (FIG. 4A). C1GALT1 catalyzes the addition of galactose to the core GalNAc-Ser/Thr residue to generate the core 1 O-glycan, a prominent mucin core structure on the endothelium (FIG. 30). C1galt1 was also chosen for knockdown because it is downregulated in brain endothelial cells in both aging and AD (FIGS. 3K-3L, 3O). Using the AAV9-derived capsid, PHP.V1, and an abbreviated CLDN5 promoter (“sCLDN5”), AAV-PHP.V1-sCLDN5::EGFP-miR-E-C1galt1 (hereafter referred to as AAV-miR-C1galt1) for brain endothelial cell-specific knock down of C1GALT1 was generated.

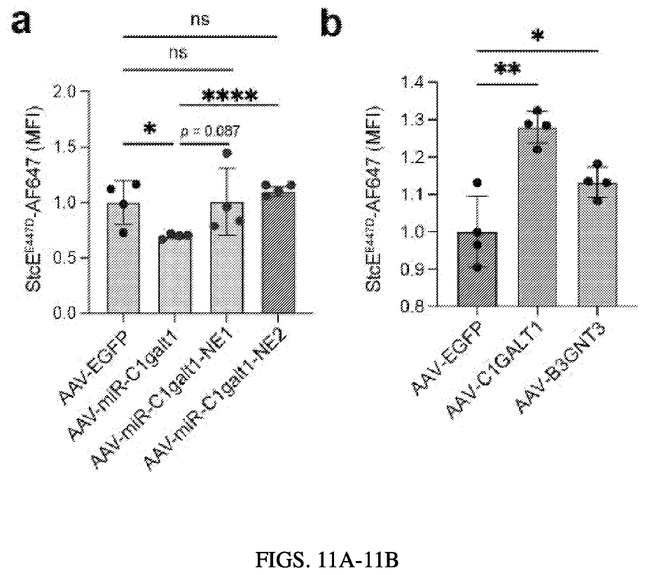

It was confirmed that AAV-miR-C1galt1 transfection of bEnd.3 cells decreased mucin domain labeling via StcE^{E447D}-AF647 compared to the control, AAV-PHP.V1-sCLDN5::EGFP (hereafter referred to as AAV-EGFP), and two less effective C1galt1-targeting miR-E constructs, AAV-miR-C1galt1-NE1 and AAV-miR-C1galt1-NE2 (Table 1; FIG. 11A). Eight weeks following intravenous injections of 3-month-old mice with AAV-miR-C1galt1 and AAV-EGFP revealed robust EGFP expression in CD31⁺ brain endothelial cells (FIG. 4F).

TABLE 1
miR-E constructs tested
for the knockdown of C1GALT1

| Plasmid | | 97-mer |
|---|---|---|
| Name | 22-nt guide | shRNA sequences |
| AAV- | TCAGCGTTGTAATA | TGCTGTTGACAGTGAGCGTCA |
| miR- | AAGTGTTA | GCGTTGTAATAAAGTGTTATA |
| E- | [SEQ ID NO: 1] | GTGAAGCCACAGATGTATAAC |
| C1galt1 | | ACTTTATTACAACGCTGGTGC |
| | | CTACTGCCTCGGA |
| | | [SEQ ID NO: 4] |
| AAV- | GACACCACTTAATC | TGCTGTTGACAGTGAGCGGAC |
| miR- | AAAGGTTA | ACCACTTAATCAAAGGTTATA |
| E- | [SEQ ID NO: 2] | GTGAAGCCACAGATGTATAAC |
| C1galt1- | | CTTTGATTAAGTGGTGTTTGC |
| NE1 | | CTACTGCCTCGGA |
| | | [SEQ ID NO: 5] |
| AAV- | TCAACATAAAGATG | TGCTGTTGACAGTGAGCGTCA |
| miR- | AGAACATA | ACATAAAGATGAGAACATATA |
| E- | [SEQ ID NO: 3] | GTGAAGCCACAGATGTATATG |
| C1galt1- | | TTCTCATCTTTATGTTGGTGC |
| NE2 | | CTACTGCCTCGGA |
| | | [SEQ ID NO: 6] |

Furthermore, AAV-miR-C1galt1 effectively reduced brain endothelial C1GALT1 levels and StcE^{E447D}-AF647 labeling compared to AAV-EGFP, to similar degrees as observed in aged mice compared to young (FIGS. 4B-4D; FIGS. 3K-3L; FIGS. 2H-2I). Using the small molecule tracer sulfo-NHS-biotin (0.5 kDa) to evaluate BBB permeability, it was observed that mice transduced with AAV-miR-C1galt1 developed leaky BBBs with numerous hotspots of sulfo-NHS-biotin leakage outside of cortical blood vessels (FIGS. 4E-4G). On the other hand, the tracer remained confined in the BBB lumens of mice transduced with AAV-EGFP, signifying their BBBs remained intact and functional (FIGS. 4E-4G). The results indicated that loss of core 1 mucin-type 0-glycans can impair BBB function.

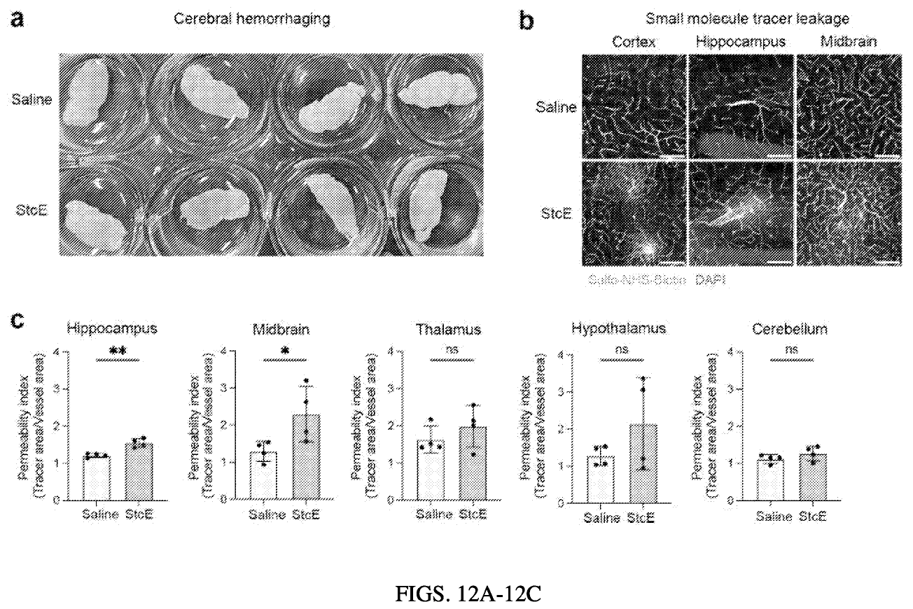

To additionally test the effects of targeted disruption of luminal mucin-type O-glycosylation, intravenous injections of StcE were used to cleave luminal mucin-domain glycoproteins in vivo. It was found that two-day intravenous injections of 0.25 mg/kg of StcE in young mice led to unexpected cerebral hemorrhaging with noticeable red blood cell leakage in the meninges and ventricles (FIGS. 4H-4J; FIG. 12A). Previously, a lethal dose of 15 mg/kg StcE in 10-week-old C57BL/6 mice also led to visible small volume hemorrhaging underneath the skull within 3 h. StcE-treated mice additionally exhibited increased BBB permeability to intravenously injected sulfo-NHS-biotin across several brain regions, including the cortex, hippocampus, and midbrain (FIGS. 4K-M; FIGS. 12B-12C). The findings suggest that luminal mucin-domain glycoproteins critically regulate BBB and vascular integrity and that their dysregulation in aging and disease may increase BBB dysfunction and susceptibility to cerebral bleeding.

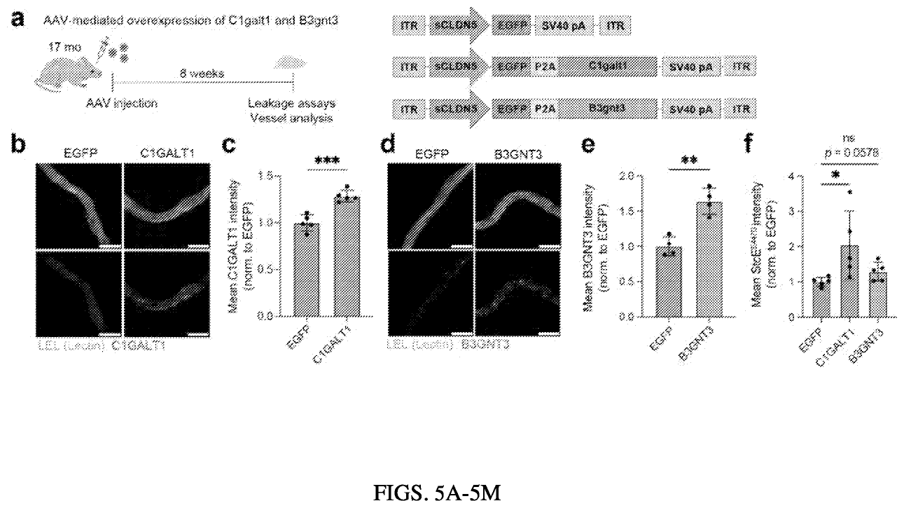

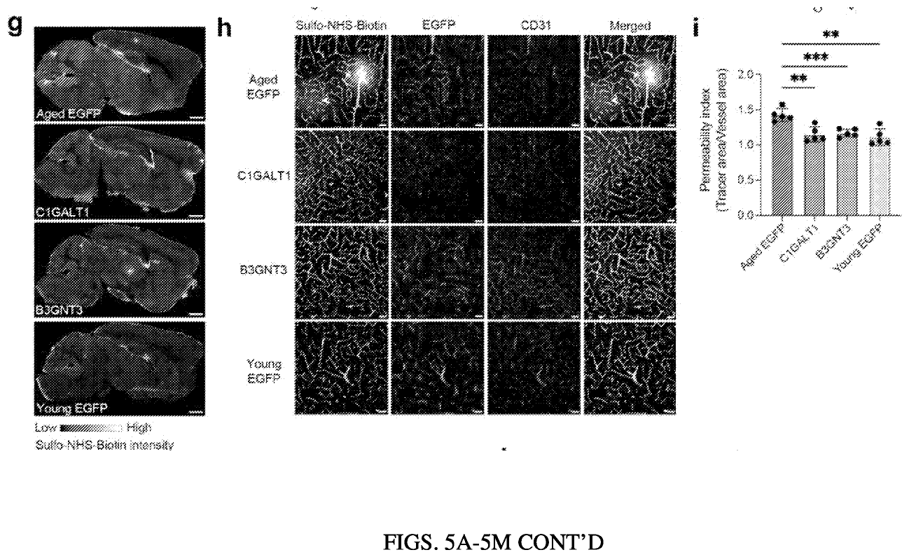

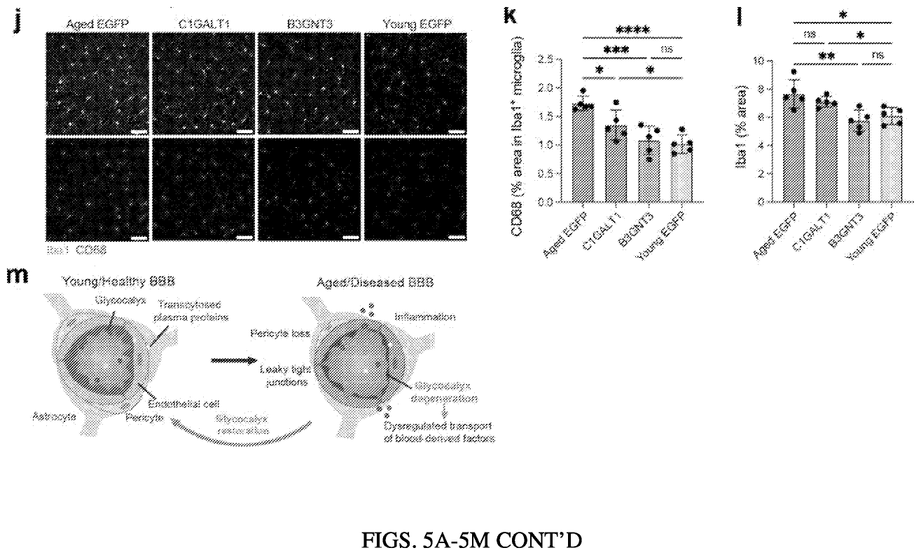

Glycocalyx restoration improves BBB function. Reversal of the detrimental effects of downregulated brain endothelial mucin-type O-glycosylation in aged mice was next tested by overexpressing two age-downregulated mucin-type O-glycan biosynthetic enzymes, C1GALT1 and B3GNT3, in brain endothelial cells (FIG. 5A; FIGS. 3K-30). Transduction of bEnd.3 cells with AAV-PHP.V1-sCLDN5::EGFP-C1GALT1 and AAV-PHP.V1-sCLDN5::EGFP-B3GNT3 (hereafter referred to as AAV-C1GALT1 and AAV-B3GNT3, respectively) increased mucin domain labeling via StcE^{E447D}-AF647 compared to transduction with AAV-EGFP (FIG. 111B).

After eight weeks post-AAV injection of 17-month-old mice, robust EGFP expression in CD31V brain endothelial cells with all constructs and increased brain endothelial cell levels of C1GALT1 and B3GNT3 with transduction of AAV-C1GALT1 and AAV-B3GNT3, was verified, respectively, compared to AAV-EGFP (FIGS. 5B-5E, 5H). Increased StcE^{E447D}-AF647 cerebrovascular labeling in mice transduced with AAV-C1GALT1 and AAV-B3GNT3 compared to AAV-EGFP was also observed (FIG. 5F).

Upon evaluation of BBB integrity in these mice, it was found that aged mice transduced with AAV-EGFP exhibited sulfo-NHS-biotin leakiness broadly across brain regions, confirming signs of BBB dysfunction expected in aged mice (FIG. 5G). However, aged mice transduced with AAV-C1 GALT1 and AAV-B3GNT3 exhibited significantly reduced sulfo-NHS-biotin leakage from blood vessels broadly across the brain, comparable to levels observed in young mice (FIGS. 5G-51).

Furthermore, aged mice transduced with AAV-C1GALT1 and AAV-B3GNT3 showed reductions in CD68 signal in Iba1⁺ microglia, an indication of microglial activation, suggesting reduced neuroinflammation in these mice (FIGS. 5J-5K). Mice transduced with AAV-B3GNT3 yielded a more significant decline in microglial CD68 signal than mice transduced with AAV-C1GALT1 and showed reduced total Iba1 signal, another marker of microglial activation, again to levels comparable to young mice (FIGS. 5J-5L). These results suggest that elongation of the core 1 O-glycan structures by B3GNT3 on brain endothelial cells has greater anti-inflammatory effects on the brain than overexpression of C1GALT1. Together, the results suggest that increasing core 1 mucin-type O-glycosylation on the brain endothelium can effectively improve BBB function and brain health in aging and age-related disorders (FIG. 5M).

Discussion Here, brain endothelial glycocalyx dysregulation was identified as a novel molecular mechanism underlying BBB dysfunction in aging and neurodegenerative disease. A conserved decline in brain endothelial mucin-type O-glycosylation in these states was observed and reduction of this modification was demonstrated to increase BBB leakiness and susceptibility to cerebral bleeding. AAV-mediated overexpression of brain endothelial core 1 mucin-type O-glycans in aged mice improved BBB integrity and reduced signs of neuroinflammation. The results demonstrated that restoring the brain endothelial glycocalyx is an effective therapeutic route to combat BBB breakdown in age-related CNS diseases (FIG. 5M).

The contributions of mucin-domain glycoproteins to BBB function are disclosed herein, although, there were many additional brain endothelial glycosylation pathways that were identified to be dysregulated during aging and neurodegenerative diseases (FIGS. 2E-2F; FIGS. 10A-10F).

### Example 3

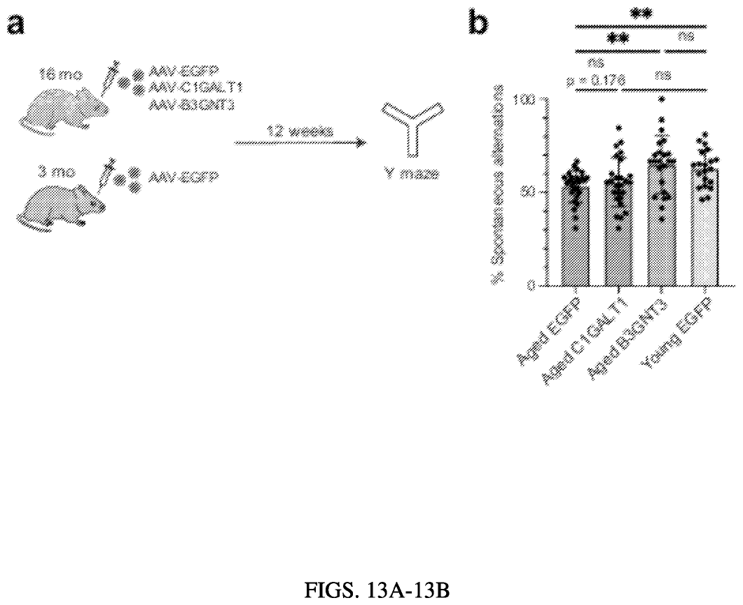

Methods. Aged mice (16-month-old) were retro-orbitally injected with AAV-EGFP, AAV-C1GALT1, or AAV-B3GNT3, and young mice (3-month-old) were retro-orbitally injected with AAV-EGFP (FIG. 13A). After 12 weeks, spatial working memory was assessed by Y Maze using the spontaneous alternation test (n=19-26 mice/group) (FIG. 13B). The Y-maze is made up of three white, opaque plastic arms at 120° angles from each other. Mice were placed in the end of one arm and allowed to freely explore all three arms for 5 min. The maze was cleaned with 70% ethanol between animals and before the first animal to eliminate traces of odor. The number of arm entries and the number of triads (a set of consecutive arm entries) were recorded in order to calculate the percentage of alternation (number of triads divided by the number of possible alternations multiplied by 100).

What is claimed is:

1. A method for treating an age-related, neurological, and vascular disease or condition comprising: administering to a subject a brain endothelial specific vector, wherein the brain endothelial specific vector encodes a mucin and/or a mucin associated protein.

2. The method of claim 1, wherein the mucin associated protein is B3GNT3.

3. The method of claim 1, wherein the mucin associated protein is C1GALT1.

4. The method of claim 1, wherein the mucin associated protein is B4GALT5, GALNT1, GALNT2, GALNT10, GALNT11, GALNT15, GALNT17, GALNT18, ST3GAL1, ST3GAL2, or ST6GALNAC4.

5. The method of claim 1, wherein the condition is one or more of Alzheimer's disease, Parkinson's disease, frontotemporal dementia, multiple sclerosis, traumatic brain injury, stroke, and/or amyotrophic lateral sclerosis.

6. The method of claim 1 wherein the vector is a viral vector, a lipid nanoparticle, or a cationic particle.

7. The method of claim 1, wherein the vector is co-administered with a targeting moiety.

8. A method for treating an age-related, neurological, and vascular disease or condition comprising: genetically modifying brain endothelial cells to express a mucin and/or a mucin associated protein.

9. The method of claim 8, wherein the mucin associated protein is B3GNT3.

10. The method of claim 8, wherein the mucin associated protein is C1GALT1.

11. The method of claim 8, wherein the mucin associated protein is B4GALT5, GALNT1, GALNT2, GALNT10, GALNT11, GALNT15, GALNT17, GALNT18, ST3GAL1, ST3GAL2, or ST6GALNAC4.

12. The method of claim 8, wherein the condition is one or more of Alzheimer's disease, Parkinson's disease, frontotemporal dementia, multiple sclerosis, traumatic brain injury, stroke, and/or amyotrophic lateral sclerosis.

13. The method of claim 8, wherein the mucin associated protein is targeted to said cells with a targeting moiety.

14. A composition comprising a brain endothelial specific vector comprising a nucleic acid that encodes a mucin associated protein.

15. The composition of claim 14, wherein the mucin associated protein is B3GNT3.

16. The composition of claim 14, wherein the mucin associated protein is C1GALT1.

17. The composition of claim 14 wherein the mucin associated protein is B4GALT5, GALNT1, GALNT2, GALNT10, GALNT11, GALNT15, GALNT17, GALNT18, ST3GAL1, ST3GAL2, or ST6GALNAC4.

18. The composition of claim 14 wherein the vector is a viral vector, a lipid nanoparticle, or a cationic particle.

19. The composition of claim 14, wherein the vector is co-administered with a targeting moiety.

20. The method or claim 5 or 8, wherein the condition is one or more of accumulation of genetic damage, reduced telomere length, epigenetic alterations in DNA, impairment of protein homeostasis, deregulated nutrient sensing, cellular senescence, stem cell exhaustion, mitochondrial dysfunction, altered cellular communication, neurodegenerative diseases, cardiovascular diseases, cerebral amyloid angiopathy, cerebrovascular diseases, cancer, autism spectrum disorder, central nervous system, immune system disorders, neurological/neurodegenerative diseases with BBB dysregulation, or musculoskeletal disorders.


SEQUENCE LISTING (6 sequences)

SEQ ID NO: 1 - DNA, 22 bp, synthetic construct
  source 1..22: mol_type = other DNA
tcagcgttgtaataaagtgtta

SEQ ID NO: 2 - DNA, 22 bp, synthetic construct
  source 1..22: mol_type = other DNA
gacaccacttaatcaaaggtta

SEQ ID NO: 3 - DNA, 22 bp, synthetic construct
  source 1..22: mol_type = other DNA
tcaacataaagatgagaacata

SEQ ID NO: 4 - DNA, 97 bp, synthetic construct
  source 1..97: mol_type = other DNA
tgctgttgacagtgagcgtcagcgttgtaataaagtgttatagtgaagccacagatgtataacactttattacaacgctggtgcctactgcctcgga

SEQ ID NO: 5 - DNA, 97 bp, synthetic construct
  source 1..97: mol_type = other DNA
tgctgttgacagtgagcggacaccacttaatcaaaggttatagtgaagccacagatgtataacctttgattaagtggtgtttgcctactgcctcgga

SEQ ID NO: 6 - DNA, 97 bp, synthetic construct
  source 1..97: mol_type = other DNA
tgctgttgacagtgagcgtcaacataaagatgagaacatatagtgaagccacagatgtatatgttctcatctttatgttggtgcctactgcctcgga